(19) United States
(12) Patent Application Publication
(10) Pub. No.: US 2024/0350503 A1
(43) Pub. Date: Oct. 24, 2024

(54) COMPOUNDS FOR TREATMENT OF VIRAL INFECTIONS BY NEUROTROPIC VIRUS

(71) Applicants: Ensieh FARAHANI, Risskov (DK); Michael Hans Willi LAPPE, Risskov (DK)

(72) Inventors: Ensieh FARAHANI, Risskov (DK); Michael Hans Willi LAPPE, Risskov (DK); Søren Riis PALUDAN, Aarhus C (DK)

(73) Assignees: Ensieh FARAHANI, Risskov (DK); Michael Hans Willi LAPPE, Risskov (DK)

(21) Appl. No.: 18/682,649

(22) PCT Filed: Aug. 17, 2022
(86) PCT No.: PCT/EP2022/072963

§ 371 Date: Feb. 9, 2024

(30) Foreign Application Priority Data
Aug. 17, 2021 (EP) 21191639.0

(51) Int. Cl. A61K 31/53; A61P 31/14; A61P 31/16; A61P 31/22

(52) U.S. Cl. CPC A61K 31/53; A61P 31/14; A61P 31/16; A61P 31/22

(57) ABSTRACT

Activators of hypoxia-inducible factor 1-alpha for treatment and prophylaxis of viral infections caused by neurotropic viruses are provided. The compounds are broad-spectrum antivirals and are effective against many different neurotropic viruses, such as herpes simplex and SARS-CoV-2.

### TECHNICAL FIELD OF THE INVENTION

The present invention relates to compounds for treatment and prevention of virus infections. In particular, the present invention relates to broad-spectrum antiviral compounds.

### BACKGROUND OF THE INVENTION

Viral infections can cause severe diseases, and efficient immune responses are required to control the invading pathogens. While the adaptive immune system is essential for clearance of infections and for lasting immunity, the innate immune system exerts the first line of defence and also primes activation of the adaptive response. In the absence of rapid and efficient innate immune responses, the subsequent immune activities mediated by secondary innate mechanisms and adaptive immune responses may amplify inflammation and promote disease. The innate immune response to viral infections is believed mainly to be driven by mechanisms activated down-stream of pattern recognition receptors (PRRs), which sense viral molecules, primarily nucleic acids, to induce host defence and inflammation. The best-described PRR-driven antiviral system is the type I interferon (IFN), which are cytokines acting in auto- and paracrine manners to induce expression of IFN-stimulated genes (ISGs) with antiviral and immune-stimulatory functions. However, viruses efficiently evade immune responses, to allow establishment and maintenance of infection. For instance, most viruses, including influenza A virus, herpes simplex virus (HSV)-1 and SARS-CoV-2 inhibit type I IFN expression and function, and most herpesviruses counteracts CD8+ T cell immunity.

HSV-1 and HSV-2 are alpha-herpesvirus, which productively infect epithelial cells and neurons. In addition, the virus can enter into other cell types, including immune cells, and modulate their functions. HSV infections can give rise to a number of severe diseases, including herpes simplex encephalitis and recurrent genital herpes. A requirement for development of these diseases is productive viral replication in permissive cells and viral modulation of host defence responses. HSV infections seem not only to modulate the abundance of specific gene transcripts, but also to cause disruption of transcription termination.

Thus, effective antivirals for treatment of HSV infections are needed and many have already been developed. For example, Jakub Treml et al. discloses a range of compounds that have been reported to possess anti-HSV activity. Antiviral compounds that are targeted specifically towards particular virus particles may however be less efficient in treating latent and recurrent virus since they can be rendered ineffective due to mutations conferring drug-resistance.

A different approach is to use compounds that does not target the virus itself but instead turns host cellular factors against the virus, thus reducing viral replication, development, activity, and/or survival. This type of antivirals are within the group of broad-spectrum antiviral compounds as they do not target a specific virus but can be used for simultaneous treatment of many different viruses and co-infections. Host cellular factors are considerably more difficult to circumvent by mutations compared to direct interactions with specific chemical compounds. Thus, using broad-spectrum antivirals is a way to avoid the emergence of drug-resistant virus strains.

Hence, an antiviral compound being less susceptible to drug-resistance caused by virus mutations would be advantageous, and in particular a more efficient and/or reliable broad-spectrum antiviral compound would be advantageous.

### SUMMARY OF THE INVENTION

Thus, an object of the present invention relates to compounds that possess antiviral activity without necessarily targeting specific virus particles. The compounds modulate host cellular factors to prevent invasion and replication of virus in general and may therefore fall within the group of broad-spectrum antivirals.

The inventors of the present invention have found that infection by neurotropic virus seems to down-regulate the HIF1-alpha transcription network in order to maintain surroundings wherein the virus can survive and replicate. The compounds are therefore activators of hypoxia-inducible factor 1-alpha (HIF1-alpha) which is a cornerstone in the HIF1-alpha transcription network.

A particular object of the present invention is therefore to provide a compound which is an activator of the hypoxia-inducible factor 1-alpha and possesses an indirect antiviral activity against neurotropic virus. The compounds are therefore considered to solve the above-mentioned problems of the prior art with treatment of latent and recurrent virus and virus that develop drug resistances.

Thus, a first aspect of the invention relates to a compound for use in the treatment or prophylaxis of a neurotropic viral infection caused by a neurotropic virus, wherein said compound is an activator of hypoxia-inducible factor 1-alpha (HIF1-alpha) of formula (I):

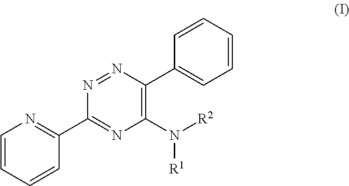

wherein
   R¹ is represented by —(CH₂)ₙ—R³, or forms together with R² a five or six-membered nitrogen containing heterocycle,
   R² is selected from the group consisting of hydrogen and C₁-C₄ alkyl, or forms together with R¹ a five- or six-membered nitrogen containing heterocycle,
   n is an integer selected from 0, 1, 2 and 3, and
   R³ is selected from the group consisting of optionally substituted phenyl, allyl, methyl, cyclopropyl, cyclobutyl, cyclopentyl, cyclohexyl, tert-butyl, tetrahydrofuranyl, tetrahydropyranyl, and piperidinyl, and
wherein the neurotropic virus is selected from the group consisting of human alphaherpesvirus 1 (HHV-1), human alphaherpesvirus 2 (HHV-2), human alphaherpesvirus 3 (HHV-3), human betaherpesvirus 5 (HHV-5), human betaherpesvirus 6A (HHV-6A), human betaherpesvirus 6B (HHV-6B), human betaherpesvirus 7 (HHV-7), human gammaherpesvirus 8 (HHV-8), human adenovirus (HAdV), Monkeypox virus Zaire-96-I-16, Human mastadenovirus, Vaccinia virus, horsepox virus HSPV050, cowpox virus, variola virus, human enterovirus, human rhinovirus, human papillomavirus, severe acute respiratory syndrome-related coronavirus 2 (SARS-CoV-2), encephalomyocarditis virus (EMCV), poliovirus, influenza A virus, influenza B virus, human immunodeficiency virus 1 (HIV-1), human immunodeficiency virus 2 (HIV-2), vesicular stomatitis Indiana virus (VSV or VSIV), and rabies lyssavirus.

### BRIEF DESCRIPTION OF THE FIGURES

FIG. 1 shows that the HIF1-alpha gene transcription measured in transcription per kilobase million (TPM) in human neuroblast cells decreases a few hours after infection by HSV-1. Results for uninfected cells (UI) is shown for comparison.

FIG. 2 shows that the HIF1-alpha gene transcription measured in transcription per kilobase million (TPM) in human neuroblast cells decreases a few hours after infection by HSV-2. Results for uninfected cells (UI) is shown for comparison.

FIG. 3A shows that the number of plaque forming units (PFU) in titer from human neuroblast cells infected with HSV-1 and treated with a HIF1-alpha activator (ML228) is significantly decreased in comparison to cells treated with the control substance.

FIG. 3B shows that HSV-1 replication in human neuroblast cells treated with a HIF1-alpha activator (ML228) is significantly decreased in comparison to cells treated with the control substance.

FIG. 4A shows that the number of plaque forming units (PFU) in titer from human neuroblast cells infected with HSV-2 and treated with a HIF1-alpha activator (ML228) is significantly decreased in comparison to cells treated with the control substance.

FIG. 4B shows that HSV-2 replication in human neuroblast cells treated with a HIF1-alpha activator (ML228) is significantly decreased in comparison to cells treated with the control substance.

FIG. 5A shows that the number of plaque forming units (PFU) in titer from human neuroblast cells infected with VSV and treated with a HIF1-alpha activator (ML228) is significantly decreased in comparison to cells treated with the control substance.

FIG. 5B shows that the number of plaque forming units (PFU) in titer from human neuroblast cells infected with EMCV and treated with a HIF1-alpha activator (ML228) is significantly decreased in comparison to cells treated with the control substance.

FIG. 6A shows that SARS-CoV-2 replication in human neuroblast cells treated with a HIF1-alpha activator (ML228) is significantly decreased in comparison to cells treated with the control substance.

FIG. 6B shows that Influenza A virus replication in human neuroblast cells treated with a HIF1-alpha activator (ML228) is significantly decreased in comparison to cells treated with the control substance.

FIG. 7A shows that the number of plaque forming units (PFU) in titer from mouse primary neurons infected with HSV-1 and treated with a HIF1-alpha activator (ML228) is significantly decreased in comparison to cells treated with the control substance.

FIG. 7B shows that HSV-1 replication in mouse primary neurons treated with a HIF1-alpha activator (ML228) is significantly decreased in comparison to cells treated with the control substance.

FIG. 8A shows that the number of plaque forming units (PFU) in titer from mouse primary neurons infected with HSV-2 and treated with a HIF1-alpha activator (ML228) is significantly decreased in comparison to cells treated with the control substance.

FIG. 8B shows that HSV-2 replication in mouse primary neurons treated with a HIF1-alpha activator (ML228) is significantly decreased in comparison to cells treated with the control substance.

The present invention will now be described in more detail in the following.

### DETAILED DESCRIPTION OF THE INVENTION

### Definitions

Prior to discussing the present invention in further details, the following terms and conventions will first be defined:

### Neurotropic Virus

In the present context, the term “neurotropic virus” refers to a virus that is capable of infecting nerve cells. A neurotropic virus may also possess other capabilities apart from being able to infect nerve cells. For example, a neurotropic virus may also be able to infecting other cell types, such as for example epithelial cells.

### HSV-(1/2)

In the present context, the term “HSV-(1/2)” refers to both HSV-1 and HSV-2 virus.

### Neurotropic Viral Infection

In the present context, the term “neurotropic viral infection” refers to a condition wherein a neurotropic virus invaded or is invading tissue and/or cells. During an infection, the virus may be replicating and in some cases, it may cause disease.

HIF1-alpha

In the present context, the term “HIF1-alpha” refers to the hypoxia-inducible factor 1-alpha gene producing the HIF1-alpha protein.

### Activator

In the present context, the term “activator” refers to a compound which activates or increases a biological process, such as the transcription activity of a gene.

### Agonist

In the present context, the term “agonist” refers to a compound or agent which is capable of binding to a receptor, thus to activate or increase a biological process, such as the transcription activity of a gene.

### Viral Down-Regulation

In the present context, the term “viral down-regulation” refers to a condition wherein a biological process is inhibited, reduced or counteracted by a virus infection.

### HIF1-Alpha Transcription Network

In the present context, the term “HIF1-alpha transcription network” refers to a biological process which is activated and partly controlled by the HIF1-alpha activity. The biological process comprises a network of chemical reaction pathways, including compounds and proteins.

### HIF PHDi

In the present context, the term “HIF PHDi” refers to a compound which can inhibit the hydroxylation activity of prolyl hydroxylase domain proteins (PHDs) and thus falls within the group of hypoxia-inducible factor-prolyl hydroxylase inhibitors (HIF PHDis).

### Neuroinvasive

In the present context, the term “neuroinvasive” refers to a virus which is capable of accessing and/or entering the nervous system.

### Neurovirulent

In the present context, the term “neurovirulent” refers to a virus which is capable of causing disease within the nervous system.

### Latency Period

In the present context the term “latency period” refers to a period in which a virus does not confer any disease conditions to the host and is unable to transmit from the host.

### Incubation Period

In the present context the term “incubation period” refers to a period in which a virus is replicating in a host, but without conferring any disease conditions to the host until the concentration of the virus reaches a high enough level.

### Disease Period

In the present context the term “disease period” refers to a period in which a virus is causing a disease in a host.

### Pharmaceutically Acceptable

In the present context, the term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

### Excipient

In the present context, the term “excipient” refers to a natural or synthetic substance formulated alongside the active or therapeutic ingredient (an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability.

### Carrier

In the present context, the term “carrier” refers to any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

### Lipid-Based Drug Delivery System

In the present context the term “lipid-based drug delivery system” refers to a formulation comprising a lipid excipient.

The present invention is based on the finding that neurotropic virus infection seems to down-regulate the HIF1-alpha transcription network as described in example 2. It was therefore considered possible that administration of a HIF1-alpha activator could be effective at treating infections caused by such virus. Examples 3 and 4 show that activators of HIF1-alpha are effective at reducing and treating infections caused by neurotropic viruses in a number of different cells obtained from mammals.

Thus, a first aspect of the present invention relates to a compound for use in the treatment or prophylaxis of a neurotropic viral infection, wherein said compound is an activator of hypoxia-inducible factor 1-alpha (HIF1-alpha).

An embodiment of the present invention relates to the compound for use as described herein, wherein viral down-regulation of the HIF1-alpha transcription network is counteracted. Another embodiment of the present invention relates to the compound for use, wherein viral down-regulation of the HIF1-alpha transcription network is inhibited. An additional embodiment of the present invention relates to the compound for use, wherein viral down-regulation of the HIF1-alpha transcription network is reduced.

The activator may work directly or indirectly to increase the activity of HIF1-alpha. For example, in some embodiments the activator is an agonist binding to a receptor whereby the HIF1-alpha transcription network is activated or the activity increased. However, in other embodiments the activator is a compound inhibiting the hypoxia-inducible factor-proline dioxygenase enzyme which otherwise breaks down or delimit the activity the HIF1-alpha transcription factors. Thus, an embodiment of the present invention relates to the compound for use as described herein, wherein the activator of hypoxia-inducible factor 1-alpha (HIF1-alpha) is selected from the group consisting of an agonist of HIF1-alpha, and a hypoxia-inducible factor-prolyl hydroxylase inhibitor (HIF PHDi).

Some compounds sharing a similar structural backbone are considered especially relevant because, these compounds seem particularly strong at counteracting neurogenic virus replication and survivability as shown in examples 3 and 4. A more specific embodiment of the present invention therefore relates to the compound for use as described herein, wherein the activator of HIF1-alpha is selected from the group consisting of a compound of formula (I):

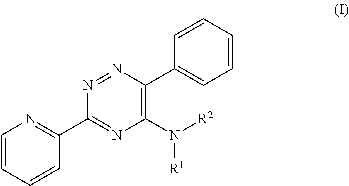

wherein
   R¹ is represented by —(CH₂)ₙ—R³, or forms together with R² a five or six-membered nitrogen containing heterocycle,
   R² is selected from the group consisting of hydrogen and C₁-C₄ alkyl, or forms together with R¹ a five- or six-membered nitrogen containing heterocycle,
   n is an integer selected from 0, 1, 2 and 3, and
   R³ is selected from the group consisting of optionally substituted phenyl, allyl, methyl, cyclopropyl, cyclobutyl, cyclopentyl, cyclohexyl, tert-butyl, tetrahydrofuranyl, tetrahydropyranyl, and piperidinyl.

For those embodiments wherein R³ is a substituted phenyl located at the end of the —(CH₂)ₙ— hydrocarbon bridge in R¹ some substituents are considered especially relevant. Accordingly, an embodiment of the present invention relates to the compound for use as described herein, wherein the optionally substituted phenyl is substituted with one or more of the substituents selected from the list consisting of phenyl, C₁-C₄ alkyl, fluoro, chloro, and C₁-C₄ alkoxy.

In another embodiment of the present invention, n is 1 (i.e. the hydrocarbon bridge conforms to a single —CH₂— group). Thus, an embodiment of the present invention relates to the compound for use as described herein, wherein the activator of HIF1-alpha is a compound of formula (II):

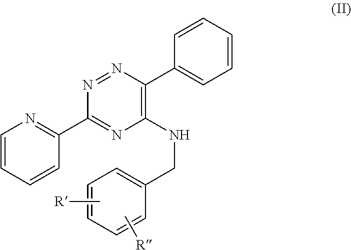

wherein
R′ is selected from the group consisting of hydrogen, phenyl, and tert-butyl, and
R″ is selected from the group consisting of hydrogen, chloro, and fluoro.

Compounds falling within the scope of formula (II) all possess the same structural backbone. However, some compounds within the scope may be pointed out in a list of particularly relevant species, wherefore, an embodiment of the present invention relates to the compound for use as described herein, wherein the activator of HIF1-alpha is selected from the group consisting of:
6-phenyl-N-[(4-phenylphenyl)methyl]-3-pyridin-2-yl-1,2,4-triazin-5-amine,
6-phenyl-N-[(4-phenyl-(3-chlorophenyl))methyl]-3-pyridin-2-yl-1,2,4-triazin-5-amine,
6-phenyl-N-[(4-phenyl-(3-fluorophenyl))methyl]-3-pyridin-2-yl-1,2,4-triazin-5-amine,
6-phenyl-N-[(4-(tert-butyl)phenyl)methyl]-3-pyridin-2-yl-1,2,4-triazin-5-amine,
6-phenyl-N-[(4-(tert-butyl)-(3-chlorophenyl))methyl]-3-pyridin-2-yl-1,2,4-triazin-5-amine, and
6-phenyl-N-[(4-(tert-butyl)-(3-fluorophenyl))methyl]-3-pyridin-2-yl-1,2,4-triazin 5-amine.

A particularly important embodiment of the present invention relates to the compound for use as described herein, wherein the activator of HIF1-alpha is a compound of formula (III):

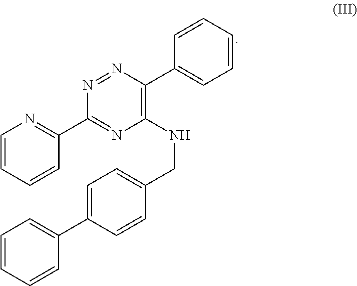 [SMILES: c1ccc(-c2ccc(CNc3nc(-c4ccccn4)nnc3-c3ccccc3)cc2)cc1]

The compound of formula (III) is referred to as ML228.

### Neurotropic Virus and Related Diseases

Infections caused by neurotropic virus has been associated with a suppression of the HIF1-alpha transcription network. This suppression is considered a means by which neurotropic virus may increase their survivability by avoiding HIF1-alpha controlled antiviral responses of the host. An embodiment of the present invention therefore relates to the compound for use as described herein, wherein the neurotropic viral infection is caused by a neurotropic virus. In this regard, the meaning of the term “caused by a neurotropic virus” may include any infection comprising at least one neurotropic virus and eventually other types of virus. A neurotropic virus is considered to be any virus capable of infecting a nerve cell. In addition, a neurotropic virus may be neuroinvasive whereby it is able to enter the nervous system and/or neurovirulent in which case it can cause a disease of the nervous system. Thus, an embodiment of the present invention relates to the compound for use as described herein, wherein the neurotropic virus is neuroinvasive and/or neurovirulent.

Neurotropic virus are mostly classified to be members of a limited number of virus families. In this regard, an embodiment of the present invention relates to the compound for use as described herein, wherein the neurotropic virus is classified as a member of a family selected from the group consisting of herpesviridae, coronaviridae, picornaviridae, orthomyxoviridae, retroviridae, rhabdoviridae, flaviviridae, togaviridae, polyomaviridae, paramyxoviridae, peribunyaviridae, and matonaviridae.

A first family specific embodiment of the present invention relates to the compound for use as described herein, wherein the neurotropic virus is classified as a member of the family of herpesviridae or coronaviridae. A second family specific embodiment of the present invention relates to the compound for use as described herein, wherein the neurotropic virus is classified as a member of the family of herpesviridae or picornaviridae. A third family specific embodiment of the present invention relates to the compound for use as described herein, wherein the neurotropic virus is classified as a member of the family of herpesviridae or retroviridae. A fourth family specific embodiment of the present invention relates to the compound for use as described herein, wherein the neurotropic virus is classified as a member of the family of herpesviridae or rhabdoviridae.

The neurotropic virus infection may be caused by genus specific neurotropic virus. An embodiment of the present invention relates to the compound for use as described herein, wherein the neurotropic virus is a member of a genus selected from the group consisting of simplexvirus, varicellovirus, lymphocryptovirus, cytomegalovirus, roseolovirus, rhadinovirus, alphacoronavirus, betacoronavirus, gammacoronavirus, deltacoronavirus, aphtovirus, avihepatovirus, cardiovirus, enteroviruses, erbovirus, hepatovirus, kobuvirus, parechovirus, tescovirus, tremovirus, sapelovirus, senecaviruses, alphainfluenzavirus, betainfluenzavirus, gammainfluenzavirus, deltainfluenzavirus, lentivirus, flavivirus, alphavirus, rubivirus, vesiculovirus, and lyssavirus.

The neurotropic virus infection may be caused by specific virus species. Another embodiment of the present invention therefore relates to the compound for use as described herein, wherein the neurotropic virus is selected from the group consisting of human alphaherpesvirus 1 (HHV-1), human alphaherpesvirus 2 (HHV-2), human alphaherpesvirus 3 (HHV-3), human herpes virus 4 (HHV-4, also known as Epstein-Barr virus (EBV)), human betaherpesvirus 5 (HHV-5), human betaherpesvirus 6A (HHV-6A), human betaherpesvirus 6B (HHV-6B), human betaherpesvirus 7 (HHV-7), human gammaherpesvirus 8 (HHV-8), human adenovirus (HAdV), monkeypox virus, Human mastadenovirus, Vaccinia virus, horsepox virus, cowpox virus, variola virus, human enterovirus, human rhinovirus, parainfluenza viruses of humans (hPIVs), respiratory syncytial virus (RSV), varicella-zoster virus, cytomegalovirus (CMV), tick-borne encephalitis viruses (TBEV), human papillomavirus, flavivirus family, wets nile virus (WNV), dengue virus (DENV), human coronavirus 229E (HCoV-229E), human coronavirus HKU1 (HCoV-HKU1), human coronavirus NL63 (HCoV-NL63), human coronavirus OC43 (HCoV-OC43), middle east respiratory syndrome-related coronavirus (MERS-CoV), severe acute respiratory syndrome-related coronavirus (SARS-CoV), severe acute respiratory syndrome-related coronavirus 2 (SARS-CoV-2), encephalomyocarditis virus (EMCV), poliovirus, influenza A virus, influenza B virus, influenza C virus, influenza D virus, human immunodeficiency virus 1 (HIV-1), human immunodeficiency virus 2 (HIV-2), zika virus, chikungunya virus, vesicular stomatitis Indiana virus (VSV or VSIV), and rabies lyssavirus.

Another embodiment of the present invention relates to the compound for use as described herein, wherein the neurotropic virus is selected from the group consisting of human alphaherpesvirus 1 (HHV-1), human alphaherpesvirus 2 (HHV-2), human alphaherpesvirus 3 (HHV-3), human betaherpesvirus 5 (HHV-5), human betaherpesvirus 6A (HHV-6A), human betaherpesvirus 6B (HHV-6B), human betaherpesvirus 7 (HHV-7), human gammaherpesvirus 8 (HHV-8), human adenovirus (HAdV), Monkeypox virus Zaire-96-I-16, Human mastadenovirus, Vaccinia virus, horsepox virus HSPV050, cowpox virus, variola virus, human enterovirus, human rhinovirus, human papillomavirus, severe acute respiratory syndrome-related coronavirus 2 (SARS-CoV-2), encephalomyocarditis virus (EMCV), poliovirus, influenza A virus, influenza B virus, human immunodeficiency virus 1 (HIV-1), human immunodeficiency virus 2 (HIV-2), vesicular stomatitis Indiana virus (VSV or VSIV), and rabies lyssavirus.

An embodiment of the present invention relates to the compound for use as described herein, wherein the neurotropic virus is selected from the group consisting of herpes simplex virus 1 (HSV-1), herpes simplex virus 2 (HSV-2), encephalomyocarditis virus (EMCV), poliovirus, severe acute respiratory syndrome-related coronavirus 2 (SARS-CoV-2), influenza A virus, influenza B virus, human immunodeficiency virus 1 (HIV-1), human immunodeficiency virus 2 (HIV-2), vesicular stomatitis Indiana virus (VSV or VSIV), and rabies lyssavirus.

Infection by a neurotropic virus may cause a disease if the virus is allowed to replicate and spread within the host. The compounds of the present invention are considered effective at inhibiting or reducing virus replication and survivability. Application of the compounds is therefore not only relevant for preventing virus replication and spreading before a disease occurs, but also for treatment of already established diseases. An embodiment of the present invention thus relates to the compound for use as described herein, wherein a disease caused by the viral infection is also treated or prevented.

An embodiment of the present invention relates to the compound for use as described herein, wherein the disease caused by the viral infection is selected from the group consisting of Alzheimer's disease, Alzheimer's disease related dementias (ADRD), Parkinson's disease, Guillain-Barre syndrome, multiple sclerosis, epilepsy, meningitis, aseptic meningitis, encephalitis, myelitis, acute disseminated encephalomyelitis, meningoencephalitis, herpes simplex encephalitis, recurrent genital herpes, varicella-zoster encephalitis, poliomyelitis, encephalomyocarditis, arthropod-borne encephalitis, subacute sclerosing panencephalitis (SSPE), progressive multifocal leukoencephalopathy (PML), flaccid paralysis, enteroviral disease, eastern equine encephalitis (EEE), western equine encephalitis, St. Louis encephalitis, rabies, La crosse encephalitis, progressive rubella panencephalitis (PRP), COVID-19, post-acute sequelae of COVID-19 (PASC), myalgic encephalomyelitis/chronic fatigue syndrome (ME/CFS), neuromyelitis optica spectrum disorder (NMOSD), dysautonomina, polyradiculitis, inflammatory neuropathies, and hypoxia.

Yet another embodiment of the present invention relates to the compound for use as described herein, wherein the disease caused by the viral infection is selected from the group consisting of Alzheimer's disease, Alzheimer's disease related dementias (ADRD), Parkinson's disease, Guillain-Barre syndrome, multiple sclerosis, epilepsy, meningitis, encephalitis, myelitis, acute disseminated encephalomyelitis, meningoencephalitis, herpes simplex encephalitis, recurrent genital herpes, varicella-zoster encephalitis, poliomyelitis, encephalomyocarditis, arthropod-borne encephalitis, COVID-19, post-acute sequelae of COVID-19 (PASC), myalgic encephalomyelitis/chronic fatigue syndrome (ME/CFS), neuromyelitis optica spectrum disorder (NMOSD), dysautonomina, polyradiculitis, inflammatory neuropathies, and hypoxia.

The compound of the present invention is preferably changing the cellular environment surrounding the virus to conditions wherein the virus replication is reduced or hindered. Thus, the compounds work around the virus and may therefore be suitable for preventing virus from replicating at any stage of an infection, such as but not limited to stages wherein the virus has small structural alterations or mutated entirely. One embodiment of the present invention therefore relates to the compound for use as described herein, wherein the compound is administered during a latency period, or during an incubation period, or during a disease period, of the viral infection.

An infection takes place in a viral host, such as in a subject wherein the virus is present. Thus, an embodiment of the present invention relates to the compound for use as described herein, wherein the treatment is for use in a subject in need thereof. In this regard, the term “subject” may comprise any animal with a HIF1-alpha gene. However, mammals seems to be particular important and an embodiment of the present invention therefore relates to the compound for use as described herein, wherein the subject is a mammal. A further embodiment of the present invention is directed to specific groups of mammals and thus relate to the compound for use as described herein, wherein the mammal is selected from the group consisting of human, pig, dog, horse, cattle, and cat; preferably a human.

An embodiment of the present invention relates to the compound for use as described herein, wherein the compound is administered to the subject by intravenous administration (IV), oral administration, intramuscular injection (IM), intrathecal administration, intraperitoneal injection (IP), and intraventricular administration. Additionally, an embodiment of the present invention relates to the compound for use as described herein, wherein the compound is administered in conjunction with at least one pharmaceutically acceptable excipient and/or pharmaceutically acceptable carrier.

An embodiment of the present invention relates to the compound for use as described herein, wherein the compound is administered in a lipid-based drug delivery systems (LBDDS). A more detailed embodiment of the present invention relates to the compound for use as described herein, wherein the compound is administered in the lipid-based drug delivery systems (LBDDS) comprising components selected from the group consisting of oils, such as triglycerides, surfactants, such as water soluble and water in-soluble surfactants, co-solvents, and water.

An embodiment of the present invention relates to the compound for use as described herein, wherein the compound is administered in an emulsion, microemulsion, liposome, oil dispersion or nanoparticle.

An embodiment of the present invention relates to the compound for use as described herein, wherein the compound is administered in a dose comprising 0.1 to 1000 μg/kg, such as 0.1 to 700 μg/kg, such as 0.1 to 500 μg/kg, such as 0.1 to 300 μg/kg, such as 0.1 to 200 μg/kg, such as 0.1 to 100 μg/kg, of the compound based on the body weight of the subject in need of treatment. A specific embodiment of the present invention relates to the compound for use as described herein, wherein the compound is administered in a dose comprising 0.1 to 100 μg/kg of the compound based on the body weight of the subject in need of treatment.

An embodiment of the present invention relates to the compound for use as described herein, wherein the compound is administered at least once or twice per week. Administration may also happen on a daily basis and an embodiment of the present invention therefore relates to the compound for use as described herein, wherein the compound is administered 1 to 4 times per day, such as once every day, such as twice every day, such 3 times per day, such 4 times per day. Yet another embodiment of the present invention relates to the compound for use as described herein, wherein the first administration is performed more than 12 hours after the neurotropic virus entered the host, such as more than 24 hours after.

An embodiment of the present invention relates to the compound for use as described herein, wherein the treatment is performed in conjunction with an additional treatment involving one or more steps selected from the group consisting of administration of intravenous fluid, symptomatic treatment, administration of an additional antiviral drug, virus vaccination, and combinations thereof. Particularly, an embodiment of the present invention relates to the compound for use as described herein, wherein the treatment is performed in conjunction with administration of one or more additional antiviral drug. The additional antiviral drug may be selected from any known and/or commercially available drug. In this regard, an embodiment of the present invention relates to the compound for use as described herein, wherein the additional antiviral drug is selected from the group consisting of aciclovir (ACV), vidarabine, valaciclovir, corticosteroids, ganciclovir, foscarnet, valganciclovir, ribavirin, brincidofovir, cidofovir, FV-100, and valomaciclovir.

A first alternative aspect of the present invention relates to a method of treating or preventing a viral infection in a subject in need thereof, the method comprising administration of a compound which is an activator of hypoxia-inducible factor 1-alpha (HIF1-alpha).

A second alternative aspect of the present invention relates to a pharmaceutical composition for use in treatment or prophylaxis of a viral infection in a subject in need thereof, the pharmaceutical composition comprising a compound which is an activator of hypoxia-inducible factor 1-alpha (HIF1-alpha).

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Cell Lines, Reagents and Culture Conditions

### Cultures of Human SH-SY5Y and HaCaT Cells.

Human neuroblastoma (SH-SY5Y) and immortalized human HaCaT keratinocytes were cultured in Dulbecco's modified eagle medium (DMEM, Gibco/Sigma) supplemented with 10% heat inactivated fetal calf serum (FCS, Sigma-aldrich), 1% penicillin/streptomycin (Gibco)+L-Glutamine (Sigma-Aldrich).

### Isolation and Culturing of Mouse Primary Neuron Cells

Mouse primary neuron cells were isolated from mice by using the Neural Tissue Dissociation Kit (MACS, Miltenyi Biotec) according to the manufacturer's instructions. The cells were preplated for 45 minutes in culture flasks to avoid the quick attachment of non-neuronal cells. The floating neurons were seeded on poly-D-lysine (Sigma-Aldrich) and laminin (Invitrogen) precoated 24-well plates. The cells were cultured in Neurobasal-A medium (Gibco) containing B-27 Supplement (Gibco), 2 mM GlutaMAX (Gibco), 100 mg/ml Primocin (Invivogen) and 20 mM floxuridine b 20 mM (Sigma). Media was changed every second day. On day 7, purity of the cells was checked by flow cytometry.

### Cultures of Vero Cells

Vero cells were cultured in DMEM supplemented with 10% heat inactivated FCS, 1% penicillin/streptomycin+L-Glutamine. ML228 (Tocris) was dissolved in DMSO (Sigma-aldrich).

### Viruses

The HSV-(1/2) viruses used for in the examples were HSV-1 KOS strain and HSV-2 333 strain. The virus were propagated in Vero cells, purified by ultra-centrifugation, and titrated by standard plaque assay. Indiana strain VSV and FA strain EMCV virus were treated similarly and applied in the experiments. The SARS-CoV-2 virus used for the examples was of the alpha variant and isolated from a patient in the UK. The virus was propagated in Vero cells and viral replication was validated by SARS-CoV-2 genome detection with Taqman based qPCR using SARS-CoV-2 specific primers and probes. Influenza virus A (H1N1) strain PR8 was propagated in SPF eggs in the allantoic cavity. The allantoic fluid was layered on sucrose after concentration and suspension in Hepes-Saline. The interface band was diluted, pelleted and resuspended in Hepes-Saline. Antigen was tested for protein concentrate of 2 mg of protein per MI using a Bio-Rad colorimetric protein assay. For influenza virus A, RNA level against matrix 2 (M2) was measured by using qPCR (Taqman) with customized primers.

### Example 2—HSV-(1/2) Infection Decreases Activity of the HIF1-Alpha Network

### Multiplexed RNA-Sequencing

SH-SY5Y and HACAT cells were seeded in 24-well culture plates (15×10⁴ cells). After 24 h, cells were washed and infected with HSV-1 at a multiplicity of infection (MOI) of 3 and HSV-2 at a MOI of 1 for 4 h, 12 h and 24 h. Supernatants were discarded post infection, and the cells were used for RNA isolation and mRNA-Seq. Two biological replicates for each experiment and uninfected cells (UI) as the negative control were included in the study. Total RNA was isolated using an RNA isolation kit (Roche) according to the manufacturer instructions. The mRNA-Sequences from the total RNA were extracted using the CLC differential expression RNA-Seq tool for each cell type infected with different viruses at different time points and were uniquely barcoded using KAPA mRNA Hyper Prep Kit (Roche). A total of 42 libraries were pooled in one sample and were sequenced on Illumina's NovaSeq-S1 sequencing platform using a paired end protocol at Aarhus University Hospital (Department of Molecular Medicine).

### Mapping of RNA-Seq Data

Sequencing data composed of four technical replicates for each library, were processed using CLC bio workbench; each time point yielded at least 44 million paired reads, which passed FASTQC quality control. The reads from each time point was first mapped against HSV-1 genome strain KOS and HSV-2 genome strain HG52 with 99% genome identity with strain 333. The reads not mapped to the virus genome were mapped against human genome (hg38) and the BAM files were exported.

### Quantification of Reads-Out and Reads-In

For the further investigations, a BED file was constructed following a protocol wherein all protein coding genes in the human genome (hg38, 21463 genes) in a window downstream of the 3′ end and upstream of the 5′-end was created. The window size was set to 5 kilo base pairs (kbp) if the adjacent intergenic region was 15 kbp or longer, or to a third of the length of the intergenic region otherwise. In case the resulting length of the window was below 100 base pairs (bp), the neighbouring genes were excluded from the analysis. Any pair of genes on the same strand with overlap between their annotated regions or adjacent regions were also removed (3578 genes), resulting in a bed file with 17885 genes and their adjacent upstream and downstream window each.

The resulting BED file was used to calculate the coverage (read counts) from the BAM files using the bed-tools utilities. Thereby we quantified the number of reads mapped against each gene as well as its upstream (reads-in) and downstream region (reads-out). Then, transcripts per million (TPM) values for the genes and the window of downstream of the 3′-end or upstream of the 5′-end of each gene were calculated at 4 h, 12 h and 24 h time points in both infected and UI conditions. The ratio of TPM reads-in or reads-out versus the TPM of the associated gene was used to quantify the extent of read-out or read-in for all conditions. The resulting read-in and read-out ratios at different time points (4 h, 12 h and 24 h post infection) were normalized versus the TPM of UI, resulting relative TPM values. If the fold change of this relative TPM value was <3 for reads-in and <5 for reads out, the gene is classified as unaffected, meaning no drastic increase in transcript-tional activity downstream or upstream of the gene during virus infection.

### Gene Expression Analysis—Identification of Up- and Down-Regulated Genes

From the read-mapping, expression values for each time point relative to the UI (uninfected) as the control group were calculated for every gene and transcript (a total of 21474 genes) using the Wald statistical test in order to test the differences between all test samples versus UI. The fold changes are calculated from a generalized linear model (GLM), which corrects differences in library size between the samples and the effects of confounding factors. The threshold for the false discovery rate (FDR) p-value was determined using Benjamini-Hochberg correction for multiple testing. In this study, thresholds of the FDR p-value 0.05 and |log 2(Fold-Change)|>=1.5 were used to define significant differentially expressed genes for further functional analysis.

### Functional Enrichment Analysis

Biological functions of transcriptionally disrupted, down-regulated and up-regulated genes in each cell type were identified through pathway enrichment analysis and the results are shown in Table 1. Only pathways with a p-value <0.05 (using Fisher exact test) which have appeared in at least two different databases in Enrichr are represented.

TABLE 1
Results of the functional enrichment analysis.

| | | |
|---|---|---|
| Duration of | | |
| infection | Regulated | Pathway enrichment |

Effects by HSV-1 infection of neuroblast cells (SH-SY5Y):

| | | |
|---|---|---|
| 4 | DOWN | Antiviral sensing and IFN signalling pathways |
| hours | DOWN | Inflammation mediated by cytokines |
| 12-24 | UP | HDACs deacetylate histones |
| hours | UP | RNA polymerase I & III Transcription |
| | UP | Oxidative stress induced Senescence |
| | DOWN | DNA Damage Response |
| | DOWN | Cell cycle & apoptosis |
| | DOWN | Hypoxia response via HIF activation |

Effects by HSV-2 infection of neuroblast cells (SH-SY5Y):

| | | |
|---|---|---|
| 4 | UP | HIF-2-alpha transcription factor network |
| hours | DOWN | Antiviral sensing and IFN signalling pathways |
| 12-24 | UP | RNA polymerase I & III Transcription |
| hours | UP | HDACs deacetylate histones |
| | DOWN | Antiviral sensing and IFN signalling pathways |
| | DOWN | HIF-1-alpha transcription factor network |
| | DOWN | Apoptosis & cell cycle |
| | DOWN | Messenger RNA splicing |
| | DOWN | DNA Damage Response |

Effects by HSV-1 infection of epithelial cells (HaCaT):

| | | |
|---|---|---|
| 12-24 | UP | Interleukin-1 regulation of extracellular matrix |
| hours | UP | MAPK signalling pathway |
| | DOWN | Oxidative stress response |
| | DOWN | Nrf2 pathway |
| | DOWN | Cell cycle & DNA repair |
| | DOWN | HIF-1-alpha transcription factor network |

### Conclusion

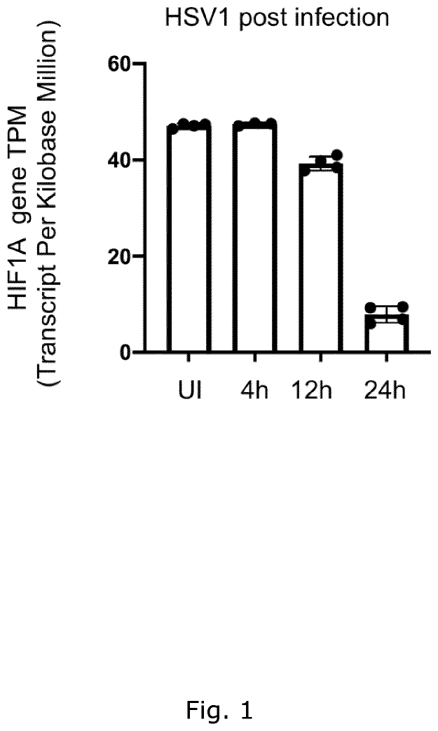

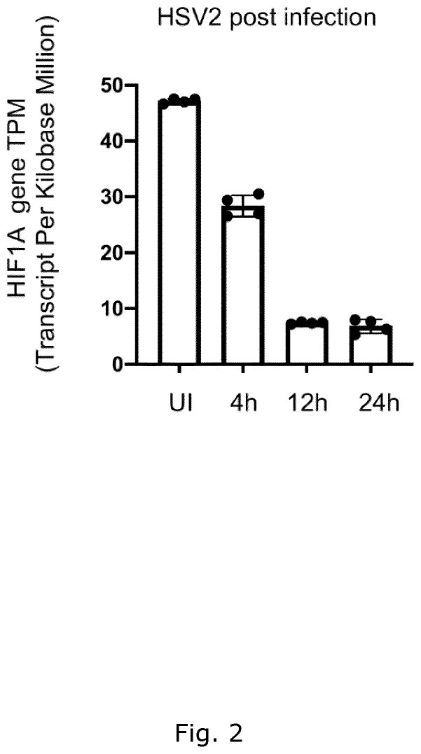

The results in Table 1 show that infections caused by neurotropic virus in neuroblast and epithelial cells changes the activity of many different biologic pathways in the cells. Notably, a decreased HIF-activation and down-regulation of the HIF1-alpha transcription factor network was identified. The decreased gene transcription in neuroblast SH-SY5Y cells infected for 4, 12 and 24 hours by neurotropic HSV-(1/2) virus, relative to uninfected cells (UI), is shown in FIG. 1 and FIG. 2, respectively. Thus, a cell infected by a neurotropic virus seems to enter a state wherein the HIF1-alpha transcription factor network is inhibited.

### Example 3—the HIF1-Alpha Activator, ML228, Counteracts Replication and Survivability of Neurotropic Virus in Human Neuroblast Cells

Human neuroblast SH-SY5Y cells (4×10⁵) were seeded in three replicates in 24 well plates. The next day, cells were pre-treated with ML228 and DMSO at a concentration of 0.5 μM for 2 hours. HSV-1, at a MOI of 0.3 and HSV-2, at a MOI of 0.1, VSV at a MOI of 0.001 and EMCV, at a MOI of 0.1 were used to infect the cells. 1 hour following the infection, the virus was removed and the cells were incubated with ML228 and DMSO for 18 hours. In the samples infected with HSV-(1/2), the supernatant was used for virus plaque assay and the cells were lysed for RNA-extraction and RT-qPCR. In the samples infected with VSV and EMCV, Supernatant was used for virus titration using TCID50% assay.

Similarly, SH-SY5Y cells (4×10⁵) were seeded in five replicates, in 24 well plates. 24 hours later, the cells were pre-treated with ML228 and DMSO at a concentration of 0.5 μM for 2 hours. The cells were infected with SARS-CoV-2 at a MOI of 1. Two hours following the infection, the virus was removed and the cells were incubated with ML228 and DMSO for 48 hours. Subsequently, the cells were lysed for RNA-extraction and RT-qPCR.

Similarly, 4×10⁵ SH-Sy5y cells were seeded in five replicates in 24 well plates. 24 hours later the cells were pretreated with ML228 and DMSO at concentration of 1 μM for 2 hours. The cells were infected with Influenza virus A at MOI of 0.1. Two hours after the infection, the virus was removed and the cells were incubated with ML228 and DMSO (1 μM) for 48 hours. Subsequently, the cells were lysed for RNA-extraction and RT-qPCR.

### Results for HSV-(1/2)

Supernatants collected from SH-SY5Y cells treated with ML228 and DMSO and infected with HSV-1 and HSV-2, were used for virus titer in a virus plaque assay. For this purpose, Vero cells at a density of 1.2×10⁶ cells/petri dish, in (DMEM) were seeded and left overnight in order to settle and to form the monolayer. The next day, cells were infected with 100 μL of supernatants in appropriate serial dilutions. The cells were incubated for 1 hour at 37° C. Subsequently, 5 ml DMEM supplemented with 0.2% normal human immunoglobin (Octapharma) were added to the cells. The plates were incubated for 2-3 days at 37° C. and stained with 0.03% methyl blue. The plaques were counted and the results were represented as PFU (Plaque-forming units)/ml in each sample.

Results for the human neuroblast cells infected with HSV-(1/2) were also obtained using reverse transcription quantitative PCR (RT-qPCR). Total RNA was extracted using the High Pure RNA Isolation kit (Roche) according to the manufacturer's instructions. RNA quality and concentration were assessed using Nanodrop spectrometry (Thermo Fisher). Viral replication was analysed by measuring RNA level against HSV-(1/2) Glycoprotein B (gB) using Brilliant III Ultra-Fast SYBR Green QRT-PCR Master Mix kit (Agilent Technology). C_{T} values were normalized versus human beta-actin gene expression in HSV-1 infection, and 18srRNA (ΔC_{T}) in HSV-2 infection.

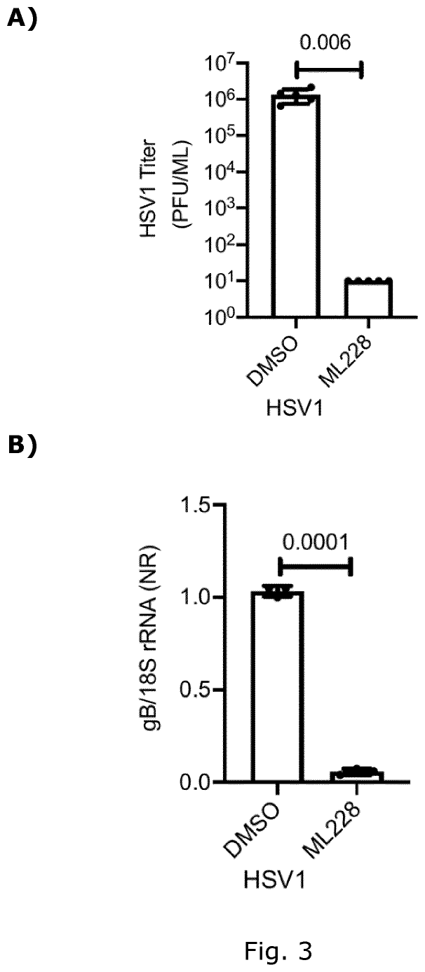

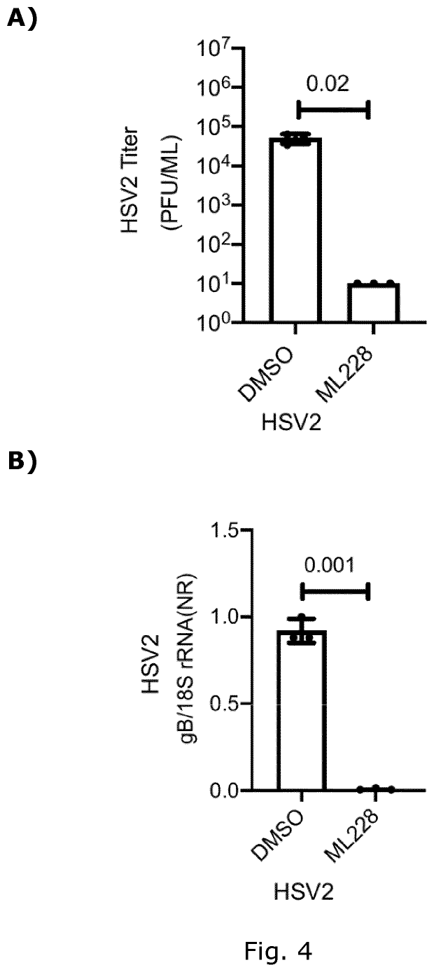

FIG. 3A and FIG. 4A show the results of the virus plaque assay of HSV-1 and HSV-2 infected human neuroblast cells, respectively. Treatment with ML228 seems to completely counteract the virus infection when compared to the control (DMSO). The same trends are observed in FIGS. 3B and 4B wherein the viral replication was determined in terms of virus RNA concentration. Thus, treatment with a HIF1-alpha activator seems effective at preventing replication of these neurotropic virus.

### Results for VSV and EMCV (TCID50% Assay)

The supernatants collected from SH-SY5Y cells treated with ML228 and DMSO and infected with VSV and EMCV, were used for virus titer. 37500 Vero cells per well were seeded in flat-bottom 96-well plates in DMEM. The following day, 10 μL of a 10-fold serial dilution of the samples were added to the cells. One full plate was used per sample replicate and each dilution were repeated 8 times on a plate. The plates were incubated for 48 hours at 37° C. and stained with 0.03% methyl blue. Virus mediated cytopathic effect was assessed by TCDI50%, calculated by the Reed-Muench method.

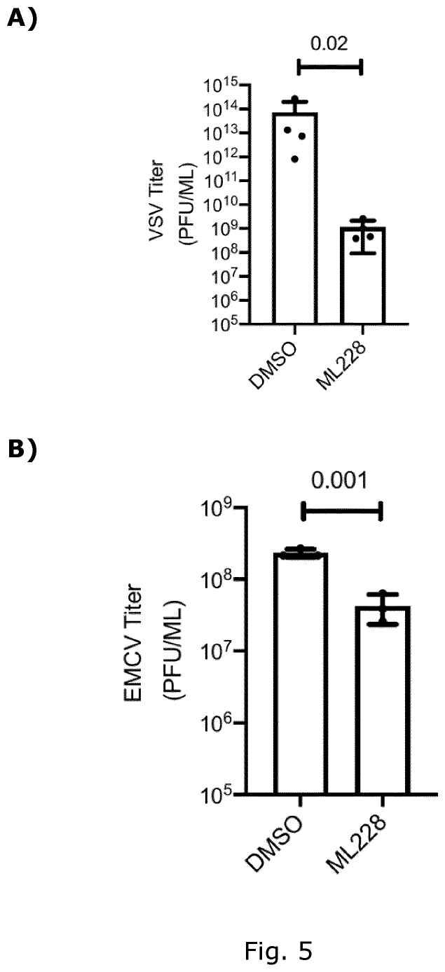

FIG. 5A shows that treatment of the VSV infected cells using ML228 significantly reduces the virus cytopathic effect when compared to the control experiment using DMSO. The same tendency is observed in FIG. 5B, wherein the results for treatment of EMCV virus with ML228 is compared to the control experiment. Thus, the HIF1-alpha activator is also able to reduce and stop infections of these neurotropic viruses.

### Results for SARS-CoV-2 and Influenza a Virus (RT-qPCR)

Total RNA was extracted using the High Pure RNA Isolation kit (Roche) according to the manufacturer's instructions. RNA quality and concentration were assessed using Nanodrop spectrometry (Thermo Fisher). For SARS-CoV-2 replication, RNA level against nucleocapsid (N2) protein was assed using Taqman based qPCR whereas RNA level against matrix 2 (M2) protein was measured using Taqman based qPCR for Influenza A virus. C_{T} values were normalized to 18srRNA (ΔC_{T}). The 18srRNA gene expression was analysed using premade TaqMan assays and the RNA-to-Ct-1-Step kit according to the manufacturer's recommendations (Applied Biosciences).

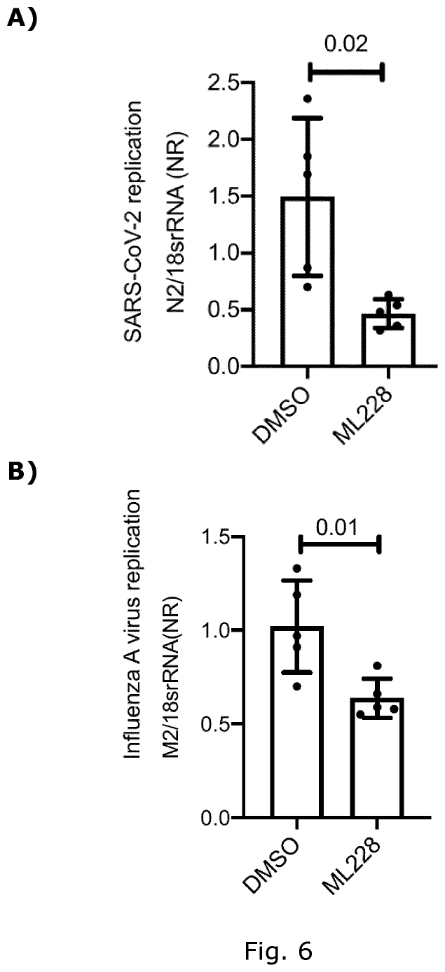

FIGS. 6A and 6B shows that an infection by SARS-CoV-2 or by Influenza A virus may be treated effectively by application of ML228. The amount of virus RNA formed in the sample comprising the HIF1-alpha activator is strongly reduced for the SARS-CoV-2 virus and also reduced for the Influenza A virus compared to the control and appears in the figures as a clear reduction of the virus replication.

### Conclusion

The general conclusion of this example is that replication of different neurotropic viruses in a range of different cell types can be significantly reduced or even stopped entirely by treating the cells with a HIF1-alpha activator (ML228).

### Example 4—HIF1-Alpha Activator, ML228, Counteracts Replication and Survivability of Neurotropic Virus in Mouse Primary Neurons

The isolated mouse primary-neurons were pre-treated with ML228 and DMSO at a concentration of 0.5 μM for 2 hours and were infected with HSV-1, at a MOI of 3 and HSV-2, at a MOI of 2. After 4 hours infection, the virus was removed and the cells were incubated with ML228 and DMSO for 72 hours. After 72 hours, the supernatant was used for virus plaque assay and the cells were lysed for RNA-extraction and RT-qPCRt.

Supernatants collected from mouse primary neurons treated with ML228 and DMSO and infected with HSV-1 and HSV-2, were used for virus titer in a virus plaque assay. For this purpose, Vero cells at a density of 1.2×10⁶ cells/petri dish, in DMEM were seeded and left overnight in order to settle and to form a monolayer. The next day, cells were infected with 100 μL of supernatant in appropriate serial dilutions. The cells were incubated for 1 hour at 37° C. Subsequently, 5 ml DMEM supplemented with 0.2% normal human immunoglobin (Octapharma) were added to the cells. The plates were incubated for 2-3 days at 37° C. and stained with 0.03% methyl blue. The plaques were counted and the results were represented as PFU (plaque-forming units) per sample.

The RT-qPCR results were obtained by total RNA extraction using the High Pure RNA Isolation kit (Roche) according to the manufacturer's instructions. RNA quality and concentration was assessed using Nanodrop spectrometry (Thermo Fisher). Viral replication was analysed by measuring RNA level against HSV-(1/2) Glycoprotein B (gB) using Brilliant III Ultra-Fast SYBR Green QRT-PCR Master Mix kit (Agilent Technology). C_{T} values were normalized versus 18srRNA (ACT) in HSV-(1/2) infection.

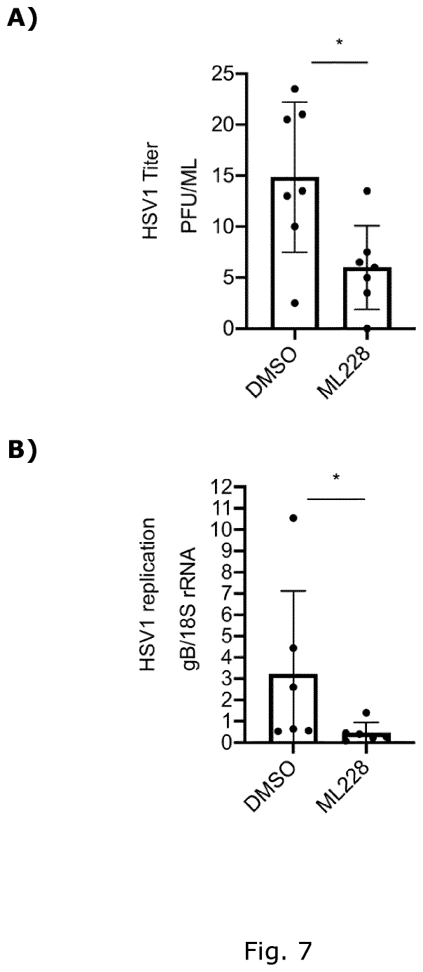

FIG. 7A shows that the number of HSV-1 plaque-forming units in the supernatants obtained from mouse primary neuron cells treated with ML228 is significantly reduced when compared to the control experiments using DMSO. The reduction of the viral replication mediated by ML228 is also illustrated in FIG. 7B, wherein the results in terms of virus RNA transcription is represented.

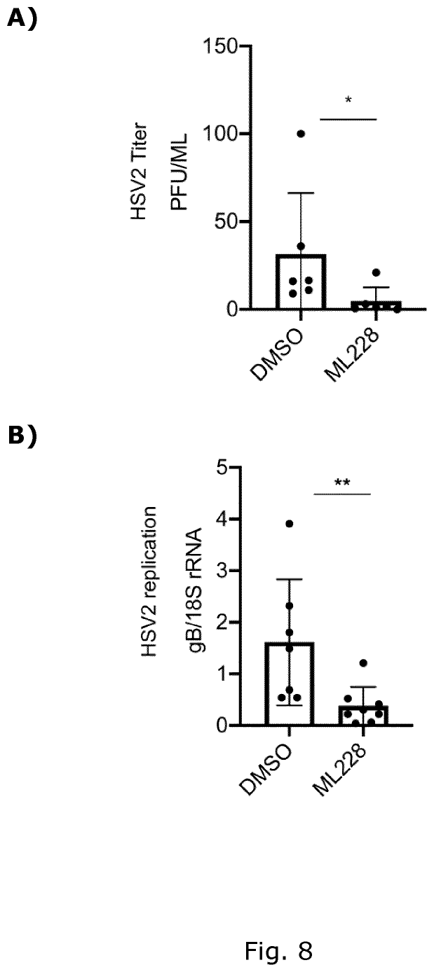

FIG. 8A shows that the HSV-2 infected mouse primary neuron cells treated with ML228 comprised significantly less plaque-forming units than the control experiments (DMSO). In addition, FIG. 8B shows that virus transcription is severely inhibited by applying ML228 to the infected cells.

### Conclusion

Application of the HIF1-alpha activator (ML228) is also able to prevent and treat infections caused by neurotropic virus in cells obtained from other mammals (mouse).

### Example 5—SH-Sy5y Cells Depleted for HIF1A, IRF1 and AAVS1 sgRNA Transfected Upon ML228 Treatment (In Vitro RNA-Seq Data Upon ML228 Treatment)

### Multiplexed RNA-Sequencing

40×10⁴ SH-Sy5y cells, depleted for HIF1A, IRF1 and AAVS1 sgRNA transfected, were seeded in 24-well culture plates. After 24 h, cells were washed and treated with ML228 and DMSO (0.5 μM) for 8 h. Supernatants were discarded post treatment, and the cells were used for RNA isolation (according to manufacturer's instructions). Samples were rRNA depleted and prepared for sequencing using SMARTer Stranded Total RNA Sample Prep Kit-HI Mammalian (Takara). For each condition, three biological replicates were included in the study. Each library was barcoded uniquely, and the libraries were pooled and sequenced as 150 bp paired-end reads on an Illumina HiSeq sequencer.

Tables 2-4 show protein-protein interactions (PPIs) between different virus proteins (listed under “Term” in said tables) and human genes in the SH-Sy5y neuroblastoma cells. The cells were either treated with ML228 or treated with DMSO (control), and the differentially expressed genes with p<0.05 and |log 2 (fold change)|>=1 from the outlined conditions were used for the in-silico host-virus protein-protein interaction analysis (P-values listed in said tables).

The number of human genes that interacts with the specific virus protein are shown in the denominator in the fractions listed under “Overlap” in said tables, whereas the numerator in the fractions indicate how many of the genes that interact with the virus protein also are induced upon ML228 treatment. The specific genes that are induced by ML228 treatment and that interact with the virus protein are listed under the “Genes” category in tables 2-4. The odds ratios (OR) listed in table 2-4 shows the association between the induction of the human genes upon treatment with ML228 and the listed virus proteins which interacts with these genes. Hence, if OR>1, the ML228 treatment increases the expression of the human genes which are associated with the shown virus proteins. Genes induced in SH-Sy5y cells by ML228 treatment relative to DMSO are in shown in Table 2. The induced human genes interacts with several viral proteins indicating that these genes might be important for the mechanistic functioning of ML228.

TABLE 2
Virus-Host-PPI for (MI228 induced genes in SH-Sy5y compared to DMSO as control)

| | | | Odds | Genes (host induced genes |
|---|---|---|---|---|
| Term | Overlap | P-value | ratio | upon ML228 treatment) |
| Orf virus ORF057 | 12/76 | 0.0018448626556509702 | 2.9491498740554154 | DUSP4; DUSP5; DUSP10; |
| putative protein- | | | | DUSP1; BAG3; DUSP19; TNNI2; |
| tyrosine | | | | DUSP26; DUSP8; DUSP6; |
| phosphatase | | | | DUSP14; DUSP7 |
| Human | 71/872 | 0.0057186355616938595 | 1.4091440469730858 | FANK1; ACVRL1; MYLK2; |
| alphaherpesvirus | | | | ANKRD37; FLT1; ANKRD33B; |
| 1 serine/threonine | | | | IRS1; SOWAHC; ZMYND8; |
| protein kinase | | | | BUB1B; MYLK3; SOX2; DUSP10; |
| US3 (gene: US3) | | | | ANKRD31; NEK2; MAP3K9; |
| | | | | PIM2; PHLDA3; DUSP4; |
| | | | | PPP1R16B; PDGFRA; MAP2K1; |
| | | | | KSR1; EPHA8; PRKCD; DYRK1B; |
| | | | | ANK3; KCNAB2; TANC2; CIT; |
| | | | | TGFBR2; CCNA1; PKM; |
| | | | | TRIB3; ULK1; PRKCQ; SGK3; |
| | | | | TNIK; TRIB2; EPHA2; CAMK2B; |
| | | | | NIM1K; BRSK2; NOTCH1; |
| | | | | GLIS3; TTK; CAMKK1; AURKA; |
| | | | | CCNB1; BAG3; CASKIN1; |
| | | | | ANKZF1; MAP2K6; NTRK2; |
| | | | | PLK3; RIOK3; NEK6; PLK2; |
| | | | | PLK1; CABLES1; BTBD11; |
| | | | | PTK6; ANKEF1; NFATC4; |
| | | | | TPCN1; MAPK10; TEC; ADCK1; |
| | | | | CCNG2; STK17B; BCL3. |
| Cercopithecine | 47/534 | 0.005798864239789109 | 1.5286195405810592 | FANK1; CAMK2B; NIM1K; |
| betaherpesvirus | | | | ANKRD37; NOTCH1; ANKRD33B; |
| 5 tegument | | | | IRS1; ZFAND2B; GLIS3; MYLK3; |
| serine/threonine | | | | CAMKK1; AURKA; CCNB1; |
| protein kinase | | | | DUSP10; BAG3; ANKRD31; |
| (gene: UL97) | | | | PPFIA4; EPHB3; PDGFRA; |
| | | | | MAP2K1; KSR1; EPHA8; PLK2; |
| | | | | NEK6; PRKCD; PLK1; DYRK1B; |
| | | | | CABLES1; PTK6; ANKEF1; |
| | | | | TANC2; CIT; TGFBR2; MAPK10; |
| | | | | CCNA1; PKM; TEC; ADCK1; |
| | | | | CCNG2; MAFF; PRKCQ; TRIB3; |
| | | | | ULK1; SGK3; GAPDH; TRIB2; |
| | | | | EPHA2. |
| Human | 71/887 | 0.008408035594460201 | 1.3820877069909236 | FANK1; ACVRL1; MYLK2; |
| herpesvirus 3 | | | | ANKRD37; FLT1; ANKRD33B; |
| strain Dumas | | | | IRS1; SOWAHC; BUB1B; MYLK3; |
| serine-threonine | | | | TRIM9; DUSP10; ANKRD31; |
| protein kinase | | | | NEK2; MAP3K9; PIM2; EPHB3; |
| | | | | DUSP4; PPP1R16B; |
| | | | | PDGFRA; MAP2K1; KSR1; |
| | | | | EPHA8; PRKCD; DYRK1B; |
| | | | | ANK3; TANC2; CIT; TGFBR2; |
| | | | | CCNA1; PKM; TRIB3; ULK1; |
| | | | | PRKCQ; SGK3; TNIK; TRIB2; |
| | | | | EPHA2; CAMK2B; NIM1K; |
| | | | | BRSK2; NOTCH1; HOXD1; |
| | | | | GLIS3; TTK; CAMKK1; AURKA; |
| | | | | CCNB1; BAG3; CASKIN1; BUB1; |
| | | | | MAP2K6; NTRK2; PLK3; |
| | | | | RASL10A; RIOK3; NEK6; PLK2; |
| | | | | PLK1; CABLES1; BTBD11; PTK6; |
| | | | | ANKEF1; NFATC4; TPCN1; TEC; |
| | | | | ADCK1; CCNG2; STK17B; |
| | | | | BCL3; MAFF. |
| Molluscum | 7/40 | 0.009104250256860841 | 3.3279618931792845 | DUSP4; DUSP10; DUSP1; |
| contagiosum | | | | DUSP8; DUSP6; DUSP14; |
| virus subtype 1 | | | | DUSP7 |
| MC082L (gene: | | | | |
| MC082L) | | | | |
| Macacine | 67/833 | 0.009276059939029175 | 1.388313398668775 | FANK1; MYLK2; FLT1; |
| alphaherpesvirus | | | | ANKRD33B; IRS1; ZMYND8; |
| 1 serine/threonine | | | | BUB1B; MYLK3; SPRED3; |
| protein kinase | | | | DUSP10; ANKRD31; NEK2; |
| US3 (gene: US3) | | | | MAP3K9; PIM2; PHLDA3; |
| | | | | PDGFRA; MAP2K1; KSR1; |
| | | | | EPHA8; PRKCD; INSRR; |
| | | | | DYRK1B; ANK3; TANC2; CIT; |
| | | | | TGFBR2; CCNA1; PKM; TRIB3; |
| | | | | ULK1; PRKCQ; SGK3; TNIK; |
| | | | | TRIB2; EPHA2; CAMK2B; |
| | | | | NIM1K; BRSK2; NOTCH1; |
| | | | | HOXD1; GLIS3; TTK; CAMKK1; |
| | | | | AURKA; CCNB1; BAG3; BUB1; |
| | | | | MAP2K6; NTRK2; PLK3; RIOK3; |
| | | | | NEK6; PLK2; PLK1; CABLES1; |
| | | | | PTK6; ANKEF1; NFATC4; TPCN1; |
| | | | | MAPK10; TEC; ADCK1; CCNG2; |
| | | | | STK17B; BCL3; MAFF; HOXD4. |
| Human | 69/868 | 0.010773375950517824 | 1.3706087636000026 | FANK1; ACVRL1; MYLK2; |
| alphaherpesvirus | | | | ANKRD37; FLT1; ANKRD33B; |
| 3 serine/threonine | | | | IRS1; SOWAHC; BUB1B; MYLK3; |
| protein kinase | | | | TRIM9; DUSP10; ANKRD31; |
| US3 (gene: ORF66) | | | | NEK2; MAP3K9; EPHB3; DUSP4; |
| | | | | PPP1R16B; PDGFRA; MAP2K1; |
| | | | | KSR1; EPHA8; PRKCD; DYRK1B; |
| | | | | ANK3; TANC2; CIT; TGFBR2; |
| | | | | CCNA1; PKM; TRIB3; ULK1; |
| | | | | PRKCQ; SGK3; TNIK; TRIB2; |
| | | | | EPHA2; CAMK2B; NIM1K; |
| | | | | BRSK2; NOTCH1; HOXD1; |
| | | | | GLIS3; TTK; CAMKK1; AURKA; |
| | | | | CCNB1; BAG3; CASKIN1; |
| | | | | MAP2K6; NTRK2; PLK3; |
| | | | | RASL10A; RIOK3; NEK6; PLK2; |
| | | | | PLK1; CABLES1; BTBD11; PTK6; |
| | | | | ANKEF1; NFATC4; TPCN1; TEC; |
| | | | | ADCK1; CCNG2; STK17B; |
| | | | | BCL3; MAFF |
| Macacine | 40/456 | 0.01088808648559931 | 1.519693762815001 | ACVRL1; MYLK2; NIM1K; |
| alphaherpesvirus | | | | BRSK2; NOTCH1; FLT1; |
| 1 tegument | | | | IRS1; GLIS3; MYLK3; CAMKK1; |
| serine/threonine | | | | AURKA; CCNB1; DUSP10; BAG3; |
| protein kinase | | | | PIM2; MAP2K6; PLK3; PDGFRA; |
| (gene: UL13) | | | | MAP2K1; RIOK3; PLK2; NEK6; |
| | | | | PRKCD; PLK1; DYRK1B; |
| | | | | CABLES1; CDC7; PTK6; ANKEF1; |
| | | | | CIT; CCNA1; PKM; TEC; ADCK1; |
| | | | | CCNG2; PRKCQ; TRIB3; SGK3; |
| | | | | TNIK; TRIB2 |
| Cowpox virus | 25/259 | 0.013458407879789669 | 1.6835539012958367 | FANK1; ANKRD37; NOTCH1; |
| CPXV213 | | | | ANKRD33B; SOWAHC; |
| protein (gene: | | | | PRICKLE2; TUBB6; PPP1R13L; |
| CPXV213 CDS) | | | | CASKIN1; ANKRD31; ANKZF1; |
| | | | | PPP1R16B; PLK2; PRKCD; PLK1; |
| | | | | BTBD11; ANK3; ANKEF1; |
| | | | | SNCAIP; MID1; TANC2; |
| | | | | TUBB2A; BCL3; PRKCQ; TNIK |
| Cowpox virus | 33/367 | 0.013946744216594243 | 1.5591355750038385 | FANK1; ANKRD37; NOTCH1; |
| CPXV016 | | | | ANKRD33B; SOWAHC; |
| protein (gene: | | | | ARHGEF28; PRICKLE2; ETS2; |
| CPXV016 CDS) | | | | RELB; PDLIM1; TUBA1C; TUBB6; |
| | | | | PDLIM2; PPP1R13L; TUBB1; |
| | | | | CASKIN1; ANKRD31; ANKZF1; |
| | | | | PPP1R16B; PLK2; PRKCD; PLK1; |
| | | | | ANKRD45; BTBD11; ANK3; |
| | | | | ANKEF1; SNCAIP; MID1; |
| | | | | TANC2; TUBB2A; BCL3; PRKCQ; |
| | | | | TNIK |
| Yaba monkey | 52/638 | 0.016077562985810867 | 1.4039905943192297 | FANK1; ANKRD37; ANKRD33B; |
| tumor virus | | | | SOWAHC; AATK; MYLK3; |
| ankyrin repeat | | | | ETS2; TUBA1C; TUBB6; |
| protein | | | | PPP1R13L; ANKRD31; NEK2; |
| | | | | PIM2; PPP1R16B; PRKCD; |
| | | | | ANKRD45; DYRK1B; ANK3; |
| | | | | MID1; TANC2; KIAA1217; |
| | | | | TUBB2A; PRKCQ; TNIK; TRIB2; |
| | | | | TRIM55; CAMK2B; NIM1K; |
| | | | | BRSK2; NOTCH1; ARHGEF28; |
| | | | | NPR2; PRICKLE2; CAMKK1; |
| | | | | RELB; AURKA; BAG3; CASKIN1; |
| | | | | ANKZF1; PLXNA3; PLK3; NTRK2; |
| | | | | PLK2; NEK6; PLK1; BTBD11; |
| | | | | PTK6; ANKEF1; SNCAIP; |
| | | | | MAPK10; STK17B; BCL3 |
| Cercopithecine | 63/816 | 0.024857384857740136 | 1.324260851331516 | FANK1; ACVRL1; MYLK2; |
| alphaherpesvirus | | | | ANKRD37; FLT1; ANKRD33B; |
| 2 serine/threonine | | | | IRS1; SOWAHC; BUB1B; MYLK3; |
| protein kinase | | | | SPRED3; TRIM9; DUSP10; |
| US3 (gene: US3) | | | | ANKRD31; NEK2; MAP3K9; |
| | | | | PIM2; PHLDA3; DUSP4; |
| | | | | PDGFRA; MAP2K1; KSR1; |
| | | | | PRKCD; DYRK1B; ANK3; |
| | | | | TANC2; CIT; TGFBR2; CCNA1; |
| | | | | PKM; TRIB3; ULK1; PRKCQ; |
| | | | | SGK3; TNIK; TRIB2; EPHA2; |
| | | | | CAMK2B; NIM1K; BRSK2; |
| | | | | NOTCH1; HOXD1; GLIS3; |
| | | | | CAMKK1; AURKA; CCNB1; |
| | | | | BAG3; MAP2K6; PLK3; RIOK3; |
| | | | | NEK6; PLK2; PLK1; CABLES1; |
| | | | | PTK6; ANKEF1; NFATC4; |
| | | | | TPCN1; TEC; ADCK1; CCNG2; |
| | | | | STK17B; MAFF |
| Monkeypox | 30/343 | 0.02572134451956831 | 1.5103404884665355 | FANK1; NIM1K; ANKRD37; |
| virus Zaire-96- | | | | NOTCH1; ANKRD33B; |
| I-16 B17R | | | | SOWAHC; PRICKLE2; ETS2; |
| (gene: B17R) | | | | TUBA1C; TUBB6; PPP1R13L; |
| | | | | TUBB1; CASKIN1; ANKRD31; |
| | | | | ANKZF1; PPP1R16B; PLK3; |
| | | | | PLK2; PRKCD; PLK1; ANKRD45; |
| | | | | BTBD11; ANK3; ANKEF1; |
| | | | | SNCAIP; TANC2; TUBB2A; |
| | | | | BCL3; PRKCQ; TNIK |
| Variola virus | 36/429 | 0.027530380991770645 | 1.4446123354068958 | FANK1; CAMK2B; NIM1K; |
| Ser/Thr kinase | | | | ANKRD37; NOTCH1; ANKRD33B; |
| (gene: C14L) | | | | IRS1; MYLK3; CAMKK1; AURKA; |
| | | | | CCNB1; BAG3; ANKRD31; PLK3; |
| | | | | PDGFRA; KSR1; EPHA8; NEK6; |
| | | | | PRKCD; PLK1; DYRK1B; |
| | | | | CABLES1; PTK6; ANKEF1; |
| | | | | TANC2; CIT; CCNA1; PKM; TEC; |
| | | | | CCNG2; PRKCQ; TRIB3; ULK1; |
| | | | | SGK3; TRIB2; EPHA2 |
| Papiine | 65/855 | 0.030415661231827963 | 1.3019232052679584 | FANK1; MYLK2; ANKRD37; |
| alphaherpesvirus | | | | FLT1; ANKRD33B; IRS1; |
| 2 serine/threonine | | | | SOWAHC; BUB1B; MYLK3; |
| protein kinase | | | | SPRED3; TRIM9; DUSP10; |
| US3 (gene: US3) | | | | ANKRD31; NEK2; MAP3K9; |
| | | | | PIM2; PHLDA3; PDGFRA; |
| | | | | MAP2K1; KSR1; PRKCD; |
| | | | | DYRK1B; ANK3; TANC2; CIT; |
| | | | | TGFBR2; CCNA1; PKM; TRIB3; |
| | | | | ULK1; PRKCQ; SGK3; TNIK; |
| | | | | TRIB2; EPHA2; CAMK2B; |
| | | | | NIM1K; BRSK2; NOTCH1; |
| | | | | HOXD1; GLIS3; TTK; CAMKK1; |
| | | | | AURKA; CCNB1; BAG3; BUB1; |
| | | | | ANKZF1; MAP2K6; NTRK2; |
| | | | | PLK3; RIOK3; NEK6; PLK2; |
| | | | | PLK1; CABLES1; PTK6; |
| | | | | ANKEF1; SNCAIP; NFATC4; |
| | | | | MAPK10; TEC; CCNG2; STK17B; |
| | | | | BCL3 |
| Vaccinia virus | 21/225 | 0.030572461185775515 | 1.6192768985766897 | FANK1; PPP1R16B; ANKRD37; |
| Ankara CP77 | | | | NOTCH1; ANKRD33B; PRKCD; |
| host range | | | | PLK1; BTBD11; ANK3; ANKEF1; |
| protein f3 | | | | SNCAIP; TANC2; TUBA1C; |
| | | | | TUBB6; TUBB2A; BAG3; |
| | | | | PPP1R13L; CASKIN1; BCL3; |
| | | | | ANKRD31; ANKZF1 |
| Human | 46/576 | 0.030587191129018407 | 1.3703005495670324 | FANK1; CAMK2B; MYLK2; |
| alphaherpesvirus | | | | NIM1K; BRSK2; NOTCH1; FLT1; |
| 3 tegument | | | | IRS1; GLIS3; MYLK3; CAMKK1; |
| serine/threonine | | | | AURKA; CCNB1; BAG3; |
| protein kinase | | | | ANKRD31; NEK2; PIM2; BUB1; |
| (gene: ORF47) | | | | MAP2K6; NTRK2; PDGFRA; |
| | | | | MAP2K1; KSR1; RIOK3; EPHA8; |
| | | | | PLK2; NEK6; PRKCD; PLK1; |
| | | | | DYRK1B; PTK6; ANK3; ANKEF1; |
| | | | | CIT; TGFBR2; PKM; TEC; |
| | | | | ADCK1; STK17B; PRKCQ; |
| | | | | TRIB3; ULK1; SGK3; TNIK; |
| | | | | TRIB2; EPHA2 |
| Orf virus | 14/134 | 0.030933590973571082 | 1.832618446874124 | PDGFRA; FLT1; LRRN3; PXDNL; |
| ORF132 | | | | NRG2; VEGFA; CLMP; BAG3; |
| vascular | | | | PDGFC; IGDCC3; CNTN2; |
| endothelial | | | | NECTIN4; MDGA1; SDK2 |
| growth factor- | | | | |
| like protein | | | | |
| Horsepox virus | 31/364 | 0.03254136172829715 | 1.4666133710502312 | FANK1; ANKRD37; NOTCH1; |
| HSPV020e | | | | ANKRD33B; SOWAHC; |
| (ankyrin repeats) | | | | PRICKLE2; ETS2; RELB; |
| | | | | AURKA; TUBA1C; TUBB6; |
| | | | | BAG3; PPP1R13L; CASKIN1; |
| | | | | ANKRD31; ANKZF1; PPP1R16B; |
| | | | | PLK3; PLK2; PRKCD; PLK1; |
| | | | | BTBD11; ETV1; ANK3; ANKEF1; |
| | | | | SNCAIP; TANC2; TUBB2A; |
| | | | | STK17B; BCL3; TRIB3 |
| Yaba monkey | 43/535 | 0.03255113329151933 | 1.3791614101485843 | FANK1; NIM1K; ANKRD37; |
| tumor virus | | | | NOTCH1; ANKRD33B; |
| ankyrin-like | | | | SOWAHC; ARHGEF28; |
| protein | | | | PRICKLE2; CAMKK1; ETS2; |
| | | | | RELB; AURKA; TUBA1C; TUBB6; |
| | | | | BAG3; PPP1R13L; TUBB1; |
| | | | | CASKIN1; ANKRD31; PIM2; |
| | | | | ANKZF1; PPP1R16B; PLK3; |
| | | | | PLK2; PRKCD; PLK1; ANKRD45; |
| | | | | DYRK1B; BTBD11; PTK6; ETV1; |
| | | | | ANK3; ANKEF1; SNCAIP; MID1; |
| | | | | TANC2; NFATC4; CIT; |
| | | | | KIAA1217; MAPK10; TUBB2A; |
| | | | | BCL3; PRKCQ |
| Human | 29/337 | 0.03358264281504959 | 1.4828342441204452 | NIM1K; BRSK2; NPR2; BUB1B; |
| gammaherpesvirus | | | | TTK; MYLK3; AURKA; CCNB1; |
| 8 ORF72 | | | | NEK2; PLK3; NTRK2; MAP2K1; |
| | | | | KSR1; EPHA8; RIOK3; PLK2; |
| | | | | PRKCD; PLK1; DYRK1B; CIT; |
| | | | | MAPK10; CCNA1; CCNG2; |
| | | | | STK17B; PRKCQ; ULK1; SGK3; |
| | | | | TNIK; EPHA2 |
| Papiine | 37/450 | 0.03361674558230061 | 1.4125152110441526 | CAMK2B; MYLK2; NIM1K; |
| alphaherpesvirus | | | | BRSK2; NOTCH1; FLT1; IRS1; |
| 2 tegument | | | | GLIS3; MYLK3; CAMKK1; |
| serine/threonine | | | | AURKA; CCNB1; BAG3; PIM2; |
| protein kinase | | | | PLK3; PDGFRA; MAP2K1; |
| (gene: UL13) | | | | RIOK3; PLK2; NEK6; PRKCD; |
| | | | | PLK1; DYRK1B; CABLES1; |
| | | | | PTK6; ANKEF1; NFATC4; CIT; |
| | | | | CCNA1; PKM; TEC; ADCK1; |
| | | | | CCNG2; PRKCQ; TRIB3; SGK3; |
| | | | | TRIB2 |
| Human | 41/508 | 0.03418982345759362 | 1.3849156184235258 | CAMK2B; MYLK2; NIM1K; |
| betaherpesvirus | | | | BRSK2; NOTCH1; FLT1; IRS1; |
| 5 tegument | | | | GLIS3; AATK; BUB1B; MYLK3; |
| serine/threonine | | | | CAMKK1; AURKA; CCNB1; |
| protein kinase | | | | BAG3; BUB1; MAP2K6; |
| (gene: UL97) | | | | MAP2K1; RIOK3; PLK2; NEK6; |
| | | | | PRKCD; PLK1; DYRK1B; |
| | | | | CABLES1; PTK6; NFATC4; CIT; |
| | | | | TGFBR2; CCNA1; PKM; TEC; |
| | | | | ADCK1; CCNG2; STK17B; |
| | | | | PRKCQ; TRIB3; ULK1; SGK3; |
| | | | | TNIK; TRIB2 |
| Human | 43/538 | 0.03518871060140894 | 1.3705781957506096 | FANK1; CAMK2B; MYLK2; |
| betaherpesvirus | | | | NIM1K; ANKRD37; NOTCH1; |
| 6 U69, | | | | FLT1; IRS1; GLIS3; AATK; |
| ganciclovir | | | | BUB1B; MYLK3; CAMKK1; |
| kinase, | | | | AURKA; TRIM9; CCNB1; BAG3; |
| phosphotransferase | | | | ANKRD31; NEK2; MAP2K6; |
| (gene: U69) | | | | PLK3; MAP2K1; KSR1; RIOK3; |
| | | | | PLK2; NEK6; PRKCD; PLK1; |
| | | | | DYRK1B; CABLES1; ANK3; |
| | | | | ANKEF1; TANC2; CIT; CCNA1; |
| | | | | TEC; ADCK1; PRKCQ; TRIB3; |
| | | | | ULK1; SGK3; TNIK; TRIB2 |
| Human | 43/538 | 0.03518871060140894 | 1.3705781957506096 | FANK1; CAMK2B; MYLK2; |
| betaherpesvirus | | | | NIM1K; ANKRD37; NOTCH1; |
| 6A tegument | | | | FLT1; IRS1; GLIS3; AATK; |
| serine/threonine | | | | BUB1B; MYLK3; CAMKK1; |
| protein kinase | | | | AURKA; TRIM9; CCNB1; BAG3; |
| (gene: U69) | | | | ANKRD31; NEK2; MAP2K6; |
| | | | | PLK3; MAP2K1; KSR1; RIOK3; |
| | | | | PLK2; NEK6; PRKCD; PLK1; |
| | | | | DYRK1B; CABLES1; ANK3; |
| | | | | ANKEF1; TANC2; CIT; CCNA1; |
| | | | | TEC; ADCK1; PRKCQ; TRIB3; |
| | | | | ULK1; SGK3; TNIK; TRIB2 |
| Horsepox virus | 9/75 | 0.03552825007421027 | 2.1392188213796253 | PPP1R16B; TUBB2A; NOTCH1; |
| HSPV020c | | | | CASKIN1; PLK1; BCL3; ANK3; |
| (Ankyrin-like | | | | ANKEF1; TANC2 |
| protein; | | | | |
| Provisional) | | | | |
| Influenza A | 13/125 | 0.03782973239051392 | 1.822516506602641 | SPTBN5; HOXD1; RNF19B; |
| virus (A/New | | | | SIX1; BARX1; SOX2; SIX6; |
| York/599/1996 | | | | PPME1; BAG3; HLX; TNNI2; |
| (H3N2)) | | | | HOXC8; DRGX |
| matrix protein | | | | |
| 1 (gene: M1) | | | | |
| Horsepox virus | 27/313 | 0.03822019861117063 | 1.4860050663622093 | FANK1; ANKRD37; NOTCH1; |
| HSPV196 | | | | ANKRD33B; PRICKLE2; ETS2; |
| (ankyrin-like | | | | TUBA1C; TUBB6; PPP1R13L; |
| protein; | | | | TUBB1; CASKIN1; ANKRD31; |
| Provisional) | | | | ANKZF1; PPP1R16B; PLK2; |
| | | | | PRKCD; PLK1; ANKRD45; |
| | | | | BTBD11; ANK3; ANKEF1; |
| | | | | SNCAIP; TANC2; TUBB2A; |
| | | | | BCL3; PRKCQ; TNIK |
| Cowpox virus | 28/328 | 0.039545135582299946 | 1.4692652482269504 | FANK1; ANKRD37; NOTCH1; |
| VHR1 or CPXV025 | | | | ANKRD33B; SOWAHC; |
| protein | | | | PRICKLE2; ETS2; RELB; |
| (gene: VHR1) | | | | TUBA1C; TUBB6; PPP1R13L; |
| | | | | TUBB1; CASKIN1; ANKRD31; |
| | | | | ANKZF1; PPP1R16B; PLK2; |
| | | | | PLK1; ANKRD45; BTBD11; |
| | | | | ANK3; ANKEF1; SNCAIP; |
| | | | | TANC2; MAPK10; TUBB2A; |
| | | | | BCL3; PRKCQ |
| Influenza A | 13/126 | 0.039958781987121675 | 1.8062913661039637 | SPTBN5; HOXD1; RNF19B; |
| virus | | | | SIX1; BARX1; SOX2; SIX6; |
| (A/Korea/426/ | | | | PPME1; BAG3; HLX; TNNI2; |
| 1968 (H2N2)) | | | | HOXC8; DRGX |
| membrane | | | | |
| protein M1 | | | | |
| (gene: M1) | | | | |
| Variola virus | 33/399 | 0.04000396823603529 | 1.4203516883844753 | FANK1; NIM1K; ANKRD37; |
| hypothetical | | | | NOTCH1; ANKRD33B; |
| protein | | | | SOWAHC; PRICKLE2; ETS2; |
| (gene: D6L) | | | | RELB; AURKA; TUBA1C; TUBB6; |
| | | | | PPP1R13L; CASKIN1; |
| | | | | ANKRD31; ANKZF1; PPP1R16B; |
| | | | | PLK3; PLK2; PRKCD; PLK1; |
| | | | | ANKRD45; BTBD11; PTK6; |
| | | | | ANK3; ANKEF1; SNCAIP; |
| | | | | TANC2; CIT; TUBB2A; BCL3; |
| | | | | PRKCQ; TNIK |
| Cowpox virus | 31/372 | 0.0417229711898555 | 1.4315854793670493 | FANK1; NIM1K; ANKRD37; |
| CPXV008 | | | | NOTCH1; ANKRD33B; |
| protein (gene: | | | | SOWAHC; PRICKLE2; ETS2; |
| CPXV008 CDS) | | | | RELB; AURKA; TUBA1C; TUBB6; |
| | | | | PPP1R13L; CASKIN1; |
| | | | | ANKRD31; ANKZF1; PPP1R16B; |
| | | | | PLK3; PLK2; PRKCD; PLK1; |
| | | | | ANKRD45; BTBD11; ANK3; |
| | | | | ANKEF1; SNCAIP; TANC2; |
| | | | | TUBB2A; BCL3; PRKCQ; TNIK |
| Monkeypox | 50/650 | 0.04423137330563385 | 1.3151922520959816 | FANK1; ANKRD37; ANKRD33B; |
| virus Zaire-96- | | | | SOWAHC; AATK; BUB1B; |
| I-16 B5R | | | | MYLK3; ETS2; TUBA1C; TUBB6; |
| (gene: B5R) | | | | PPP1R13L; TUBB1; ANKRD31; |
| | | | | NEK2; PIM2; PPP1R16B; |
| | | | | PRKCD; ANKRD45; INSRR; |
| | | | | ETV1; ANK3; MID1; TANC2; |
| | | | | CIT; TUBB2A; PRKCQ; TRIB3; |
| | | | | TNIK; TRIM55; CAMK2B; |
| | | | | NIM1K; BRSK2; NOTCH1; |
| | | | | ARHGEF28; NPR2; PRICKLE2; |
| | | | | AURKA; BAG3; CASKIN1; |
| | | | | ANKZF1; PLK2; NEK6; PLK1; |
| | | | | BTBD11; PTK6; ANKEF1; |
| | | | | SNCAIP; MAPK10; STK17B; |
| | | | | BCL3 |
| Vaccinia virus | 32/389 | 0.0452812010098185 | 1.4115159300270066 | FANK1; NIM1K; ANKRD37; |
| Ankara 72.4k | | | | NOTCH1; ANKRD33B; |
| ankyrin repeat | | | | SOWAHC; PRICKLE2; RELB; |
| protein f2 | | | | AURKA; TUBA1C; TUBB6; |
| | | | | BAG3; CASKIN1; ANKRD31; |
| | | | | PIM2; ANKZF1; PPP1R16B; |
| | | | | PLK3; PLK2; NEK6; PRKCD; |
| | | | | PLK1; BTBD11; ANK3; MIF; |
| | | | | ANKEF1; SNCAIP; TANC2; |
| | | | | TUBB2A; BCL3; PRKCQ; TNIK |
| Orf virus | 42/534 | 0.04586220056589956 | 1.3459276065628873 | FANK1; NIM1K; ANKRD37; |
| ORF129 ankyrin | | | | NOTCH1; ANKRD33B; |
| repeat protein | | | | SOWAHC; ARHGEF28; |
| | | | | PRICKLE2; CAMKK1; ETS2; |
| | | | | RELB; AURKA; PDLIM1; |
| | | | | TUBA1C; GNA14; TUBB6; |
| | | | | PPP1R13L; TUBB1; CASKIN1; |
| | | | | ANKRD31; ANKZF1; PPP1R16B; |
| | | | | PLK3; PLK2; PRKCD; PLK1; |
| | | | | ANKRD45; BTBD11; PTK6; |
| | | | | ANK3; ANKEF1; SNCAIP; MID1; |
| | | | | TANC2; NFATC4; CIT; |
| | | | | KIAA1217; MAPK10; TUBB2A; |
| | | | | BCL3; PRKCQ; TRIB2 |
| Cercopithecine | 31/377 | 0.0483700174539539 | 1.4105155950995285 | MYLK2; NIM1K; NOTCH1; FLT1; |
| alphaherpesvirus | | | | IRS1; GLIS3; CAMKK1; MYLK3; |
| 2 tegument | | | | AURKA; CCNB1; BAG3; |
| serine/threonine | | | | MAP2K6; MAP2K1; RIOK3; |
| protein kinase | | | | PLK2; NEK6; PRKCD; PLK1; |
| (gene: UL13) | | | | DYRK1B; CABLES1; PTK6; CIT; |
| | | | | CCNA1; PKM; TEC; ADCK1; |
| | | | | CCNG2; PRKCQ; TRIB3; SGK3; |
| | | | | TRIB2 |
| Cowpox virus | 24/279 | 0.049754576260339654 | 1.4801776181210398 | FANK1; PPP1R16B; ANKRD37; |
| CPXV011 | | | | NOTCH1; ANKRD33B; |
| protein (gene: | | | | SOWAHC; PLK2; ARHGEF28; |
| CPXV011 CDS) | | | | PLK1; PRICKLE2; BTBD11; |
| | | | | ANK3; ANKEF1; SNCAIP; ETS2; |
| | | | | TANC2; RELB; TUBB6; TUBB2A; |
| | | | | PPP1R13L; CASKIN1; BCL3; |
| | | | | ANKRD31; ANKZF1 |
| Monkeypox | 12/118 | 0.05165036978377601 | 1.7766265861888693 | MAP2K1; CCNB1; PKM; IRS1; |
| virus Zaire-96- | | | | BAG3; PRKCD; PLK1; PRKCQ; |
| I-16 B11R | | | | TRIB3; TNIK; MAP2K6; AURKA |
| (gene: B11R) | | | | |
| Variola virus | 27/323 | 0.05274103942100943 | 1.4350265444015444 | FANK1; ANKRD37; NOTCH1; |
| hypothetical | | | | ANKRD33B; SOWAHC; |
| protein | | | | PRICKLE2; ETS2; TUBA1C; |
| (gene: B21R) | | | | TUBB6; PPP1R13L; CASKIN1; |
| | | | | ANKRD31; ANKZF1; PPP1R16B; |
| | | | | PLK2; PRKCD; PLK1; ANKRD45; |
| | | | | BTBD11; ANK3; ANKEF1; |
| | | | | SNCAIP; TANC2; TUBB2A; |
| | | | | BCL3; PRKCQ; TNIK |
| Yaba-like | 34/424 | 0.05387249790637326 | 1.3727226864951416 | FANK1; ANKRD37; NOTCH1; |
| disease virus | | | | ANKRD33B; SOWAHC; |
| 8L protein | | | | ARHGEF28; PRICKLE2; ETS2; |
| (gene: 8L) | | | | RELB; AURKA; TUBA1C; TUBB6; |
| | | | | BAG3; PPP1R13L; TUBB1; |
| | | | | CASKIN1; ANKRD31; ANKZF1; |
| | | | | PPP1R16B; PLK3; PLK2; PRKCD; |
| | | | | PLK1; ANKRD45; BTBD11; |
| | | | | ANK3; ANKEF1; SNCAIP; |
| | | | | TANC2; KIAA1217; TUBB2A; |
| | | | | BCL3; PRKCQ; TNIK |
| Vaccinia virus | 22/255 | 0.05682339746837167 | 1.4841863118837968 | FANK1; PPP1R16B; ANKRD37; |
| Ankara CP77 | | | | NOTCH1; ANKRD33B; |
| host range | | | | SOWAHC; PRKCD; PLK1; |
| protein f2 | | | | PRICKLE2; BTBD11; ANKEF1; |
| | | | | TANC2; TUBA1C; TUBB6; |
| | | | | TUBB2A; BAG3; CASKIN1; |
| | | | | BCL3; ANKRD31; TRIB3; TNIK; |
| | | | | ANKZF1 |
| Vaccinia virus | 9/82 | 0.057458815568703095 | 1.9333654574241068 | CCNB1; PKM; IRS1; BAG3; |
| Ankara serine/ | | | | PRKCD; PLK1; PRKCQ; TRIB3; |
| threonine-protein | | | | AURKA |
| kinase-like | | | | |

ML228 is a HIF1-alpha activator. The inventors therefore investigated what genes are induced in SH-Sy5y cells with HIF1-alpha knockout (or AAVS1 knockout as a control). Genes dependent on HIF1-alpha from the transcriptome analysis in SH-Sy5y cells treated with gRNAs against HIF1-alpha and AAVS1 upon ML228 treatment are in Table 3.

TABLE 3
Virus-Host-PPI data for (SH-Sy5y cells treated with gRNAs
against HIF1A and AAVS1 as control upon ML228 treatment)

| | | | | Genes (host induced |
|---|---|---|---|---|
| | | | | genes upon ML228 |
| Term | Overlap | P-value | Odds ratio | treatment) |
| Human | 7/206 | 0.023336974285211 | 2.5823856122718856 | MYO1E; TEC; |
| immunodeficiency | | | | ARHGEF37; SH3D21; |
| virus nef protein | | | | PPP1R13L; NEDD9; |
| (gene: nef) | | | | SORBS2 |
| Human | 4/81 | 0.02491214195238195 | 3.7947182928595566 | MYO1E; ARHGEF37; |
| immunodeficiency | | | | SH3D21; SORBS2 |
| virus 1 Nef | | | | |
| (gene: nef) | | | | |
| Human | 4/105 | 0.05580228685121321 | 2.889469616106592 | TEC; SH3D21; NEDD9; |
| immunodeficiency | | | | SORBS2 |
| virus 2 nef | | | | |
| protein | | | | |
| Cercopithecine | 4/107 | 0.059014460926807205 | 2.83307467427004 | CACNB2; TEC; |
| betaherpesvirus | | | | SH3D21; SORBS2 |
| 5 membrane | | | | |
| glycoprotein | | | | |
| US2 (gene: US2) | | | | |
| Cowpox virus | 7/261 | 0.06759246720460882 | 2.017509324492333 | FANK1; MAPK7; |
| CPXV027 | | | | PPP1R13L; BCL3; |
| protein (gene: | | | | PRICKLE2; PLA2G6; |
| CPXV027 CDS) | | | | ANKZF1 |
| Human rhinovirus | 1/6 | 0.07916478228350031 | 14.501470588235295 | VLDLR |
| A30 1A | | | | |
| Cowpox virus | 7/279 | 0.08880777888014618 | 1.8822561919504643 | FANK1; MAPK7; |
| CPXV011 | | | | PPP1R13L; BCL3; |
| protein (gene: | | | | PRICKLE2; PLA2G6; |
| CPXV011 CDS) | | | | ANKZF1 |
| Cowpox virus | 7/279 | 0.08880777888014618 | 1.8822561919504643 | FANK1; MAPK7; |
| CPXV011 | | | | PPP1R13L; BCL3; |
| protein (gene: | | | | PRICKLE2; PLA2G6; |
| CPXV011 CDS) | | | | ANKZF1 |
| Human rhinovirus | 1/7 | 0.09173793246551754 | 12.083946078431373 | VLDLR |
| A23 2A (P2-A) | | | | |
| Human rhinovirus | 1/7 | 0.09173793246551754 | 12.083946078431373 | VLDLR |
| A30 2A | | | | |
| Human rhinovirus | 1/7 | 0.09173793246551754 | 12.083946078431373 | VLDLR |
| A49 3C | | | | |
| Human | 2/39 | 0.09900413835088152 | 3.927396030717064 | ESRRG; PPARG |
| papillomavirus | | | | |
| type 72b E7 | | | | |
| protein (gene: E7) | | | | |

IRF1 is an antiviral gene that is induced upon HIF1-alpha activation (ML228 treatment), which was demonstrated in an experiment comparing the IRF1 gene expression in SH-Sy5y cells treated with AAVS1 (sgRNA) upon ML228 or DMSO (control) treatment (log 2 fold change=0.684185030469305, P-value=0.000243711653837743, adjusted p-value (padj)=0.00146219836939938). Since, HIF1A activation using ML228 induces IRF1 expression, the inventors removed IRF1 expression using gRNAs targeting IRF1 (and gRNAs targeting AAVS1 as a control) to investigate what IRF1-dependent genes are induced upon ML228 treatment. Genes dependent on IRF1 from the transcriptome analysis in SH-Sy5y cells treated with gRNAs against IRF1 and AAVS1 (control) upon ML228 treatment are in Table 4. These genes are associated with the outlined virus proteins in table 4.

TABLE 4
Virus-Host-PPI data for (SH-Sy5y cells treated with gRNAs
against IRF1 and AAVS1 as control upon ML228 treatment)

| | | | Odds | Genes (host induced genes |
|---|---|---|---|---|
| Term | Overlap | P-value | ratio | upon ML228 treatment) |
| Human | 28/607 | 3.469372027029933E−5 | 2.4931874880061407 | ROBO2; CD274; NRP1; SNED1; |
| adenovirus 31 | | | | NCAN; STMN4; FGFRL1; |
| cr1 beta protein | | | | MYOM2; ICAM1; RAB26; |
| (gene: E3 cr1 | | | | SCN1B; UNC5A; F10; KCND3; |
| beta) | | | | KCNB1; ITGA2; PXDNL; L1CAM; |
| | | | | HSPG2; PTPRB; NFASC; FMNL1; |
| | | | | COL6A1; IGDCC3; FAS; ULBP1; |
| | | | | FGFR2; NECTIN1 |
| Human | 20/439 | 5.310295092619977E−4 | 2.4289232288573905 | NTRK1; ROBO2; CD274; |
| betaherpesvirus | | | | PDGFRA; UNC5A; NCAN; |
| 6 U85, OX-2 | | | | ITGA2; STMN4; PXDNL; HSPG2; |
| homologue | | | | FGFRL1; MYOM2; ICAM1; |
| (gene: U85) | | | | PTPRB; FMNL1; IGDCC3; |
| | | | | ULBP1; SCN1B; FGFR2; |
| | | | | NECTIN1 |
| Human | 20/439 | 5.310295092619977E−4 | 2.4289232288573905 | NTRK1; ROBO2; CD274; |
| betaherpesvirus | | | | PDGFRA; UNC5A; NCAN; |
| 6A membrane | | | | ITGA2; STMN4; PXDNL; HSPG2; |
| glycoprotein | | | | FGFRL1; MYOM2; ICAM1; |
| UL119 | | | | PTPRB; FMNL1; IGDCC3; |
| (gene: U85) | | | | ULBP1; SCN1B; FGFR2; |
| | | | | NECTIN1 |
| Human | 24/598 | 9.335376428398076E−4 | 2.1330767578070264 | ROBO2; CD274; NRP1; |
| adenovirus 18 | | | | PDGFRA; UNC5A; ITGB5; |
| CR1-beta | | | | KCND3; NCAN; ITGA2; STMN4; |
| protein | | | | PXDNL; L1CAM; HSPG2; |
| (gene: E3) | | | | FGFRL1; MYOM2; ICAM1; |
| | | | | PTPRB; FMNL1; COL6A1; |
| | | | | IGDCC3; ULBP1; SCN1B; |
| | | | | FGFR2; NECTIN1 |
| Monkeypox | 26/702 | 0.001821141489421333 | 1.9621604810284055 | CPNE9; ROBO2; CD274; |
| virus Zaire- | | | | SNED1; ITGB5; NCAN; ITGB3; |
| 96-I-16 B2R | | | | STMN4; FGFRL1; ICAM1; |
| (gene: B2R) | | | | RAB26; SCN1B; NTRK1; |
| | | | | PDGFRA; UNC5A; F10; ITGA2; |
| | | | | PXDNL; HSPG2; PTPRB; FMNL1; |
| | | | | COL6A1; IGDCC3; FAS; ULBP1; |
| | | | | DDR2 |
| Human | 11/196 | 0.0019236747092590055 | 2.9911496989217197 | ROBO2; FMNL1; UNC5A; |
| mastadenovirus | | | | IGDCC3; PXDNL; L1CAM; |
| A E3 CR1- | | | | HSPG2; FGFRL1; RYR3; FGFR2; |
| alpha1 | | | | ICAM1 |
| Human | 18/425 | 0.0022247714260945567 | 2.2400082980557916 | NTRK1; CD274; SNED1; |
| betaherpesvirus | | | | UNC5A; STMN4; PXDNL; |
| 7 membrane | | | | L1CAM; HSPG2; FGFRL1; |
| glycoprotein | | | | ICAM1; PTPRB; NFASC; FMNL1; |
| UL119 (gene: | | | | IGDCC3; ULBP1; SCN1B; |
| U85) | | | | FGFR2; NECTIN1 |
| Vaccinia virus | 18/439 | 0.0031402992311437734 | 2.1639393578550883 | PRKCG; NTRK1; ANKRD29; |
| Ser/Thr kinase | | | | PDGFRA; SRMS; NOTCH1; |
| (gene: F10L) | | | | ANKRD33B; DAPK2; LMTK3; |
| | | | | PTK6; PLA2G6; FMNL1; ESPNL; |
| | | | | ADGRG2; PBK; CDK5R2; |
| | | | | FGFR2; EPHA2 |
| Cercopithecine | 9/159 | 0.004567641648402439 | 3.0081958762886596 | NTRK1; SNED1; PTPRB; |
| betaherpesvirus | | | | IGDCC3; PXDNL; L1CAM; |
| 5 membrane | | | | HSPG2; FGFR2; NECTIN1 |
| protein RL11M | | | | |
| (gene: RL11M) | | | | |
| Human | 10/207 | 0.00859015305263247 | 2.545416387937932 | ROBO2; GNAO1; PTPRB; |
| adenovirus 19 | | | | FMNL1; NFASC; UNC5A; |
| CR1-beta | | | | IGDCC3; HSPG2; FGFRL1; |
| (gene: E3) | | | | ICAM1 |
| Horsepox virus | 16/414 | 0.00895562326141105 | 2.026404990833432 | PRKCG; NTRK1; PDGFRA; |
| HSPV050 | | | | SRMS; NOTCH1; DAPK2; |
| (Ser/Thr kinase; | | | | LMTK3; PTK6; PLA2G6; |
| Provisional) | | | | TUBA1C; ESPNL; PBK; PRKG1; |
| | | | | CDK5R2; FGFR2; EPHA2 |
| Vaccinia virus | 16/414 | 0.00895562326141105 | 2.026404990833432 | PRKCG; NTRK1; PDGFRA; |
| Ankara | | | | SRMS; NOTCH1; DAPK2; |
| Serine/threonine- | | | | LMTK3; PTK6; PLA2G6; |
| protein kinase 2 | | | | TUBA1C; ESPNL; PBK; PRKG1; |
| | | | | CDK5R2; FGFR2; EPHA2 |
| Horsepox virus | 4/42 | 0.00944437688557832 | 5.2403910539708045 | SEMA5A; NOTCH1; SEMA3C; |
| HSPV161 | | | | PLXNA4 |
| (semaphorin- | | | | |
| like protein; | | | | |
| Provisional) | | | | |
| Cercopithecine | 3/23 | 0.010237042097429603 | 7.455456852791878 | F10; PXDNL; PTGS2 |
| betaherpesvirus | | | | |
| 5 prostaglandin | | | | |
| G/H synthase 2 | | | | |
| (gene: COX2) | | | | |
| Vaccinia virus | 3/23 | 0.010237042097429603 | 7.455456852791878 | SEMA5A; SEMA3C; PLXNA4 |
| semaphorin-like | | | | |
| protein (gene: | | | | |
| A39R) | | | | |
| Human | 20/576 | 0.011536668884305023 | 1.8173577848596454 | PRKCG; CDKL2; NTRK1; |
| alphaherpesvirus | | | | PDGFRA; SRMS; NOTCH1; |
| 3 tegument | | | | DAPK2; MOK; LMTK3; PTK6; |
| serine/threonine | | | | ANKEF1; PLA2G6; PLEKHA7; |
| protein kinase | | | | FMNL1; ADGRG2; PRKG1; |
| (gene: ORF47) | | | | CDK5R2; FGFR2; EPHA2; DDR2 |
| Cowpox virus | 14/356 | 0.012154657931040041 | 2.0586474890446307 | ANKRD29; NOTCH1; |
| CPXV019 | | | | ANKRD33B; PRICKLE2; |
| protein (gene: | | | | ANKEF1; PLA2G6; CLIP3; |
| CPXV019 CDS) | | | | TUBA1C; ESPNL; TUBB1; BCL3; |
| | | | | ANKRD60; PRKG1; DDR2 |
| Variola virus | 16/429 | 0.01226260377625637 | 1.951281513539621 | PRKCG; CDKL2; ANKRD29; |
| Ser/Thr kinase | | | | PDGFRA; SRMS; NOTCH1; |
| (gene: C14L) | | | | ANKRD33B; DAPK2; LMTK3; |
| | | | | PTK6; ANKEF1; PLA2G6; |
| | | | | ESPNL; CDK5R2; FGFR2; EPHA2 |
| Monkeypox | 18/512 | 0.014331022091473062 | 1.8371486866140387 | PRKCG; CDKL2; NTRK1; |
| virus ser/thr | | | | ANKRD29; SRMS; NOTCH1; |
| kinase | | | | ANKRD33B; DAPK2; LMTK3; |
| | | | | STMN4; PTK6; ANKEF1; |
| | | | | PLA2G6; PLEKHA7; ESPNL; |
| | | | | CDK5R2; FGFR2; EPHA2 |
| Human | 27/872 | 0.015413148422239198 | 1.619913641452103 | NOTCH1; ANKRD33B; PLA2G6; |
| alphaherpesvirus | | | | ESPNL; |
| 1 serine/threonine | | | | ADGRG2; PBK; PRKG1; CHGB; |
| protein kinase | | | | PRKCG; CDKL2; NTRK1; |
| US3 (gene: US3) | | | | ANKRD29; PDGFRA; SRMS; |
| | | | | DAPK2; LMTK3; PTK6; KCNAB2; |
| | | | | ANKEF1; PLEKHA7; CLIP3; |
| | | | | BCL3; ANKRD60; CDK5R2; |
| | | | | FGFR2; EPHA2; DDR2 |
| Ectromelia virus | 17/482 | 0.016548441189803816 | 1.8412337294850027 | PRKCG; CDKL2; NTRK1; |
| ERPV serine/ | | | | ANKRD29; PDGFRA; SRMS; |
| threonine kinase | | | | NOTCH1; ANKRD33B; DAPK2; |
| | | | | PTK6; PLA2G6; ESPNL; PBK; |
| | | | | CDK5R2; FGFR2; EPHA2; DDR2 |
| Ectromelia virus | 17/482 | 0.016548441189803816 | 1.8412337294850027 | PRKCG; CDKL2; NTRK1; |
| ERPV serine/ | | | | ANKRD29; PDGFRA; SRMS; |
| threonine kinase | | | | NOTCH1; ANKRD33B; DAPK2; |
| | | | | PTK6; PLA2G6; ESPNL; PBK; |
| | | | | CDK5R2; FGFR2; EPHA2; DDR2 |
| Vaccinia virus | 16/446 | 0.017095952820865137 | 1.8724775682109505 | CD274; F10; CSF1; ITGB5; |
| Ankara | | | | ITGB3; ITGA2; ICAM1; FMNL1; |
| Haemagglutinin | | | | RAB26; COL6A1; FAS; DTL; |
| | | | | SCN1B; FGFR2; NECTIN1; |
| | | | | DDR2 |
| Human | 3/28 | 0.01760707969305242 | 5.962842639593909 | NOTCH1; TK1; ANKEF1 |
| enterovirus | | | | |
| C118 2C | | | | |
| Papiine | 16/450 | 0.018422066649939905 | 1.8548326620462765 | PRKCG; CDKL2; NTRK1; |
| alphaherpesvirus | | | | PDGFRA; SRMS; NOTCH1; |
| 2 tegument | | | | DAPK2; LMTK3; PTK6; ANKEF1; |
| serine/threonine | | | | PLEKHA7; FMNL1; ADGRG2; |
| protein kinase | | | | PRKG1; CDK5R2; FGFR2 |
| (gene: UL13) | | | | |
| Human | 27/887 | 0.018771299210305546 | 1.5903865493400378 | NOTCH1; ANKRD33B; PLA2G6; |
| herpesvirus 3 | | | | ESPNL; |
| strain Dumas | | | | ADGRG2; PBK; PRKG1; PRKCG; |
| serine-threonine | | | | CDKL2; NTRK1; ANKRD29; |
| protein kinase | | | | PDGFRA; SRMS; DAPK2; MOK; |
| | | | | LMTK3; PTK6; ANKEF1; |
| | | | | PLEKHA7; CLIP3; FMNL1; BCL3; |
| | | | | ANKRD60; CDK5R2; FGFR2; |
| | | | | EPHA2; DDR2 |
| Cowpox virus | 13/341 | 0.01929587362279529 | 1.9894483612804879 | CDKL2; ANKRD29; NOTCH1; |
| CPXV017 | | | | ANKRD33B; PRICKLE2; |
| protein (gene: | | | | ANKEF1; PLA2G6; CLIP3; |
| CPXV017 CDS) | | | | TUBA1C; ESPNL; BCL3; |
| | | | | ANKRD60; PRKG1 |
| Human | 10/236 | 0.019915964080775724 | 2.2154764354805514 | CLIP3; ANKRD29; NOTCH1; |
| adenovirus 5 | | | | ESPNL; ANKRD33B; BCL3; |
| fiber | | | | ANKEF1; PLA2G6; ANKRD60; |
| | | | | NECTIN1 |
| Monkeypox | 13/343 | 0.020134331419126534 | 1.9771859217171717 | ANKRD29; NOTCH1; |
| virus Zaire-96- | | | | ANKRD33B; PRICKLE2; |
| I-16 B17R | | | | ANKEF1; PLA2G6; CLIP3; |
| (gene: B17R) | | | | TUBA1C; ESPNL; TUBB1; BCL3; |
| | | | | ANKRD60; DDR2 |
| Horsepox virus | 12/308 | 0.02068565898782677 | 2.033029133029133 | CLIP3; TUBA1C; ANKRD29; |
| HSPV004 | | | | NOTCH1; ESPNL; ANKRD33B; |
| (ankyrin | | | | TUBB1; BCL3; PRICKLE2; |
| repeats) | | | | ANKEF1; PLA2G6; ANKRD60 |
| Cercopithecine | 18/534 | 0.020941437220504174 | 1.7567957292753267 | PRKCG; CDKL2; NTRK1; |
| betaherpesvirus | | | | ANKRD29; PDGFRA; SRMS; |
| 5 tegument | | | | NOTCH1; ANKRD33B; DAPK2; |
| serine/threonine | | | | LMTK3; PTK6; ANKEF1; |
| protein kinase | | | | PLA2G6; ESPNL; PRKG1; |
| (gene: UL97) | | | | CDK5R2; FGFR2; EPHA2 |
| Vaccinia virus | 3/30 | 0.021196875009034832 | 5.52058657642414 | SEMA5A; SEMA3C; PLXNA4 |
| Ankara | | | | |
| Semaphorin-like | | | | |
| protein f1 | | | | |
| Horsepox virus | 18/538 | 0.022361022917462267 | 1.74291658209864 | CD274; UNC5A; F10; CSF1; |
| HSPV177 | | | | ITGB5; ITGB3; ITGA2; STMN4; |
| (Immunoglobulin | | | | VWA5B2; ICAM1; FMNL1; |
| like) | | | | RAB26; COL6A1; FAS; DTL; |
| | | | | FGFR2; NECTIN1; DDR2 |
| Horsepox virus | 18/538 | 0.022361022917462267 | 1.74291658209864 | CD274; UNC5A; F10; CSF1; |
| HSPV177 | | | | ITGB5; ITGB3; ITGA2; STMN4; |
| (Immunoglobulin | | | | VWA5B2; ICAM1; FMNL1; |
| like) | | | | RAB26; COL6A1; FAS; DTL; |
| | | | | FGFR2; NECTIN1; DDR2 |
| Horsepox virus | 12/313 | 0.02309428920510822 | 1.9987401303015921 | CLIP3; TUBA1C; ANKRD29; |
| HSPV196 | | | | NOTCH1; ESPNL; ANKRD33B; |
| (ankyrin-like | | | | TUBB1; BCL3; PRICKLE2; |
| protein; | | | | ANKEF1; PLA2G6; ANKRD60 |
| Provisional) | | | | |
| Monkeypox | 3/31 | 0.02313060169322479 | 5.323150833937636 | F10; SERPINE1; SERPINI1 |
| virus Zaire-96- | | | | |
| I-16 serine | | | | |
| protease | | | | |
| inhibitor-like | | | | |
| protein (gene: | | | | |
| C2L) | | | | |
| Vaccinia virus | 12/316 | 0.02463535353484328 | 1.978708133971292 | CLIP3; TUBA1C; ANKRD29; |
| Ankara CP77 | | | | NOTCH1; ESPNL; ANKRD33B; |
| Host range | | | | BCL3; PRICKLE2; ANKEF1; |
| protein f1 | | | | PLA2G6; ANKRD60; PRKG1 |
| Human | 26/868 | 0.024864209716737487 | 1.561504824221626 | NOTCH1; ANKRD33B; PLA2G6; |
| alphaherpesvirus | | | | ESPNL; ADGRG2; PBK; PRKG1; |
| 3 serine/threonine | | | | PRKCG; CDKL2; NTRK1; |
| protein kinase | | | | ANKRD29; PDGFRA; SRMS; |
| US3 (gene: ORF66) | | | | DAPK2; MOK; LMTK3; PTK6; |
| | | | | ANKEF1; PLEKHA7; CLIP3; |
| | | | | BCL3; ANKRD60; CDK5R2; |
| | | | | FGFR2; EPHA2; DDR2 |
| Monkeypox | 13/355 | 0.02575057558531679 | 1.9066231115984404 | CDKL2; ANKRD29; NOTCH1; |
| virus Zaire-96- | | | | ANKRD33B; PRICKLE2; |
| I-16 ankyrin- | | | | ANKEF1; PLA2G6; CLIP3; |
| like protein | | | | TUBA1C; ESPNL; TUBB1; BCL3; |
| (gene: D9L) | | | | ANKRD60 |
| Horsepox virus | 11/285 | 0.02766435364147612 | 2.0103154192352783 | CLIP3; TUBA1C; ANKRD29; |
| HSPV020b | | | | NOTCH1; ESPNL; ANKRD33B; |
| (ankyrin repeats) | | | | TUBB1; BCL3; ANKEF1; |
| | | | | PLA2G6; ANKRD60 |
| Horsepox virus | 10/250 | 0.028211644361785887 | 2.0847329888027564 | CDKL2; TUBA1C; ANKRD29; |
| HSPV035 | | | | NOTCH1; ESPNL; ANKRD33B; |
| (ankyrin repeats) | | | | BCL3; ANKEF1; PLA2G6; |
| | | | | ANKRD60 |
| Vaccinia virus | 10/252 | 0.029571248992756417 | 2.0672902385375957 | CDKL2; TUBA1C; ANKRD29; |
| ankyrin-like | | | | NOTCH1; ESPNL; ANKRD33B; |
| protein (gene: K1L) | | | | BCL3; ANKEF1; PLA2G6; |
| | | | | ANKRD60 |
| Cowpox virus | 12/328 | 0.031559867406833135 | 1.90238369225711 | CLIP3; TUBA1C; ANKRD29; |
| VHR1 or | | | | NOTCH1; ESPNL; ANKRD33B; |
| CPXV025 | | | | TUBB1; BCL3; PRICKLE2; |
| protein (gene: VHR1) | | | | ANKEF1; PLA2G6; ANKRD60 |
| Vaccinia virus | 10/255 | 0.031696801925556586 | 2.041660074882666 | TUBA1C; ANKRD29; NOTCH1; |
| Ankara CP77 | | | | ESPNL; ANKRD33B; BCL3; |
| host range | | | | PRICKLE2; ANKEF1; PLA2G6; |
| protein f2 | | | | ANKRD60 |
| Human | 23/767 | 0.03318011160493007 | 1.5588414984762233 | PRKCG; CDKL2; NTRK1; |
| alphaherpesvirus | | | | ANKRD29; PDGFRA; SRMS; |
| 2 serine/threonine | | | | NOTCH1; DAPK2; LMTK3; PTK6; |
| protein kinase | | | | PLA2G6; PLEKHA7; CLIP3; |
| US3 (gene: US3) | | | | FMNL1; ESPNL; ADGRG2; PBK; |
| | | | | PRKG1; CDK5R2; FGFR2; |
| | | | | CHGB; EPHA2; DDR2 |
| Cowpox virus | 10/259 | 0.03469526172350595 | 2.008447225594886 | CLIP3; ANKRD29; NOTCH1; |
| CPXV213 | | | | ESPNL; ANKRD33B; BCL3; |
| protein (gene: | | | | PRICKLE2; ANKEF1; PLA2G6; |
| CPXV213 CDS) | | | | ANKRD60 |
| Vaccinia virus | 10/259 | 0.03469526172350595 | 2.008447225594886 | CDKL2; TUBA1C; ANKRD29; |
| Ankara Host | | | | NOTCH1; ESPNL; ANKRD33B; |
| range protein | | | | BCL3; ANKEF1; PLA2G6; |
| | | | | ANKRD60 |
| Papiine | 25/855 | 0.0351440659616567 | 1.5200317398626766 | NOTCH1; ANKRD33B; PLA2G6; |
| alphaherpesvirus | | | | ESPNL; ADGRG2; PBK; PRKG1; |
| 2 serine/threonine | | | | PRKCG; CDKL2; NTRK1; |
| protein kinase | | | | ANKRD29; PDGFRA; SRMS; |
| US3 (gene: US3) | | | | DAPK2; LMTK3; PTK6; ANKEF1; |
| | | | | PLEKHA7; CLIP3; FMNL1; BCL3; |
| | | | | CDK5R2; FGFR2; EPHA2; DDR2 |
| Vaccinia virus | 9/225 | 0.036245280235462314 | 2.0819372852233675 | TUBA1C; ANKRD29; NOTCH1; |
| Ankara CP77 | | | | ESPNL; ANKRD33B; BCL3; |
| host range | | | | ANKEF1; PLA2G6; ANKRD60 |
| protein f3 | | | | |
| Vaccinia virus | 9/225 | 0.036245280235462314 | 2.0819372852233675 | TUBA1C; ANKRD29; NOTCH1; |
| Ankara CP77 | | | | ESPNL; ANKRD33B; BCL3; |
| host range | | | | ANKEF1; PLA2G6; ANKRD60 |
| protein f3 | | | | |
| Human | 9/227 | 0.038005461973487586 | 2.062624136952615 | CLIP3; ANKRD29; NOTCH1; |
| adenovirus E4 | | | | ESPNL; ANKRD33B; BCL3; |
| fiber protein | | | | ANKEF1; PLA2G6; ANKRD60 |
| Human | 9/227 | 0.038005461973487586 | 2.062624136952615 | CLIP3; ANKRD29; NOTCH1; |
| mastadenovirus | | | | ESPNL; ANKRD33B; BCL3; |
| E fiber (gene: L5) | | | | ANKEF1; PLA2G6; ANKRD60 |
| Vaccinia virus | 18/576 | 0.03975896045419801 | 1.6209890203421569 | CD274; F10; CSF1; ITGB5; |
| hemagglutinin | | | | NCAN; ITGB3; ITGA2; STMN4; |
| (gene: A56R) | | | | VWA5B2; ICAM1; PTPRB; |
| | | | | FMNL1; RAB26; COL6A1; FAS; |
| | | | | DTL; FGFR2; DDR2 |
| Vaccinia virus | 18/576 | 0.03975896045419801 | 1.6209890203421569 | CD274; F10; CSF1; ITGB5; |
| hemagglutinin | | | | NCAN; ITGB3; ITGA2; STMN4; |
| (gene: A56R) | | | | VWA5B2; ICAM1; PTPRB; |
| | | | | FMNL1; RAB26; COL6A1; FAS; |
| | | | | DTL; FGFR2; DDR2 |
| Horsepox virus | 14/417 | 0.03979088840243163 | 1.741507881489352 | ANKRD29; NOTCH1; |
| HSPV012 | | | | ANKRD33B; PRICKLE2; PTK6; |
| (ankyrin-like | | | | ANKEF1; PLA2G6; CLIP3; |
| protein; | | | | TUBA1C; ESPNL; BCL3; |
| Provisional) | | | | ANKRD60; PRKG1; DDR2 |
| Vaccinia virus | 11/306 | 0.042775588117004944 | 1.8651795907613946 | CLIP3; TUBA1C; ANKRD29; |
| Ankara CP77 | | | | NOTCH1; ESPNL; ANKRD33B; |
| host range | | | | BCL3; PRICKLE2; ANKEF1; |
| protein f4 | | | | PLA2G6; ANKRD60 |
| Human | 13/383 | 0.04321548704509158 | 1.7597761824324325 | SNED1; UNC5A; ITGB5; NCAN; |
| betaherpesvirus | | | | STMN4; HSPG2; FGFRL1; |
| 6B glycoprotein | | | | ICAM1; PTPRB; FMNL1; ULBP1; |
| (gene: U85) | | | | SCN1B; NECTIN1 |
| Macacine | 24/833 | 0.0443041974991082 | 1.4947656558091444 | PRKCG; CDKL2; NTRK1; |
| alphaherpesvirus | | | | ANKRD29; PDGFRA; SRMS; |
| 1 serine/threonine | | | | NOTCH1; ANKRD33B; DAPK2; |
| protein kinase | | | | LMTK3; PTK6; ANKEF1; |
| US3 (gene: US3) | | | | PLA2G6; PLEKHA7; ESPNL; |
| | | | | ADGRG2; BCL3; PBK; PRKG1; |
| | | | | CDK5R2; FGFR2; CHGB; |
| | | | | EPHA2; DDR2 |
| Vaccinia virus | 14/424 | 0.044641999485476154 | 1.711150735528243 | ANKRD29; NOTCH1; |
| hypothetical | | | | ANKRD33B; PRICKLE2; |
| protein | | | | KLHDC8B; ANKEF1; PLA2G6; |
| | | | | CLIP3; TUBA1C; FMNL1; ESPNL; |
| | | | | BCL3; ENC1; ANKRD60 |
| Cercopithecine | 3/40 | 0.0446610118613177 | 4.026478254904651 | PDGFRA; NFASC; TNFRSF11A |
| betaherpesvirus | | | | |
| 5 membrane | | | | |
| glycoprotein | | | | |
| UL144 (gene: | | | | |
| UL144) | | | | |
| Horsepox virus | 11/314 | 0.04979680182391124 | 1.8151815181518152 | CLIP3; TUBA1C; ANKRD29; |
| HSPV014b | | | | NOTCH1; ESPNL; ANKRD33B; |
| (ankyrin repeats) | | | | MSX2; BCL3; ANKEF1; PLA2G6; |
| | | | | ANKRD60 |
| Enterovirus B | 3/42 | 0.05042927773785496 | 3.8196017180788755 | NOTCH1; SEZ6L2; ICAM1 |
| 1B(VP2) | | | | |
| Orf virus ORF132 | 6/134 | 0.051142064057973675 | 2.334758631713555 | PDGFRA; NFASC; IGDCC3; |
| vascular | | | | PXDNL; FGFRL1; FGFR2 |
| endothelial | | | | |
| growth factor- | | | | |
| like protein | | | | |
| Horsepox virus | 13/394 | 0.05193823439666195 | 1.7079915791776028 | ANKRD29; NOTCH1; |
| HSPV033b | | | | ANKRD33B; PRICKLE2; |
| (Ankyrin repeats) | | | | ANKEF1; PLA2G6; CLIP3; |
| | | | | TUBA1C; ESPNL; TUBB1; BCL3; |
| | | | | ANKRD60; PRKG1 |
| Cowpox virus | 9/241 | 0.05197073550810771 | 1.9367556878777106 | TUBA1C; ANKRD29; NOTCH1; |
| CPXV041 | | | | ESPNL; ANKRD33B; BCL3; |
| protein (gene: | | | | ANKEF1; PLA2G6; ANKRD60 |
| CPXV041 CDS) | | | | |
| Molluscum | 14/434 | 0.05227309530937062 | 1.669538729329852 | COLEC12; CD274; UNC5A; |
| contagiosum | | | | NCAN; LMTK3; PXDNL; HSPG2; |
| virus subtype 1 | | | | FGFRL1; MYOM2; PLEKHA7; |
| MC080R (gene: | | | | FMNL1; ADAMTSL4; ULBP1; |
| MC080R) | | | | HBEGF |
| Cowpox virus | 10/279 | 0.052669741332551735 | 1.8571991201022064 | CLIP3; ANKRD29; NOTCH1; |
| CPXV011 | | | | ESPNL; ANKRD33B; BCL3; |
| protein (gene: | | | | PRICKLE2; ANKEF1; PLA2G6; |
| CPXV011 CDS) | | | | ANKRD60 |
| Horsepox virus | 13/396 | 0.05364500393839697 | 1.698895778938207 | ANKRD29; NOTCH1; |
| HSPV033c | | | | ANKRD33B; PRICKLE2; PTK6; |
| (ankyrin repeats) | | | | ANKEF1; PLA2G6; CLIP3; |
| | | | | TUBA1C; ESPNL; BCL3; |
| | | | | ANKRD60; DDR2 |
| Rhinovirus C | 2/19 | 0.053812011822321586 | 5.8335070737155625 | SEZ6L2; ICAM1 |
| capsid protein | | | | |
| VP2 | | | | |
| Human | 8/206 | 0.05382100562485492 | 2.0155280309521957 | ROBO2; NTRK1; PDGFRA; |
| adenovirus D37 | | | | FMNL1; UNC5A; IGDCC3; |
| 48.9 kDa (gene: E3) | | | | HSPG2; FGFRL1 |
| Human | 9/244 | 0.05534900491033961 | 1.911735029611757 | CD274; PTPRB; FMNL1; NFASC; |
| adenovirus 52 | | | | NCAN; ITGA2; STMN4; FGFRL1; |
| E3 CR1-beta1 | | | | ICAM1 |
| (gene: E3) | | | | |
| Variola virus | 13/398 | 0.05538970136107476 | 1.6898944805194804 | CD274; SNED1; UNC5A; CSF1; |
| hypothetical | | | | ITGB5; ITGA2; STMN4; ICAM1; |
| protein (gene: J9R) | | | | PTPRB; NFASC; RAB26; ULBP1; |
| | | | | NECTIN1 |
| Yaba-like | 11/320 | 0.05554842829269943 | 1.779381927326995 | CLIP3; TUBA1C; ANKRD29; |
| disease virus | | | | NOTCH1; ESPNL; ANKRD33B; |
| 146R protein | | | | BCL3; PRICKLE2; ANKEF1; |
| (gene: 146R) | | | | PLA2G6; ANKRD60 |
| Horsepox virus | 10/282 | 0.05581485219693463 | 1.8364303085575315 | CLIP3; ANKRD29; NOTCH1; |
| HSPV022 | | | | ESPNL; ANKRD33B; BCL3; |
| (ankyrin-like | | | | PRICKLE2; ANKEF1; PLA2G6; |
| protein; | | | | ANKRD60 |
| Provisional) | | | | |
| Human | 7/172 | 0.05620171882059558 | 2.1144677544677544 | ROBO2; FMNL1; UNC5A; |
| adenovirus D8 | | | | IGDCC3; PXDNL; HSPG2; |
| 45.5kDa protein | | | | FGFRL1 |
| (gene: E3) | | | | |
| Variola virus | 13/399 | 0.056276359634373024 | 1.68542881044905 | ANKRD29; NOTCH1; |
| hypothetical | | | | ANKRD33B; PRICKLE2; PTK6; |
| protein (gene: D6L) | | | | ANKEF1; PLA2G6; CLIP3; |
| | | | | TUBA1C; ESPNL; BCL3; |
| | | | | ANKRD60; DDR2 |
| Vaccinia virus | 13/401 | 0.0580784616052717 | 1.6765665270618557 | ANKRD29; NOTCH1; |
| Ankara 54.4k | | | | ANKRD33B; PRICKLE2; |
| ankyrin repeat | | | | ANKEF1; PLA2G6; CLIP3; |
| protein f1 | | | | TUBA1C; ESPNL; BCL3; |
| | | | | ANKRD60; PRKG1; DDR2 |
| Variola virus | 11/323 | 0.05858387203765153 | 1.761998472166866 | CLIP3; TUBA1C; ANKRD29; |
| hypothetical | | | | NOTCH1; ESPNL; ANKRD33B; |
| protein (gene: B21R) | | | | BCL3; PRICKLE2; ANKEF1; |
| | | | | PLA2G6; ANKRD60 |
| Horsepox virus | 9/247 | 0.05886691271761689 | 1.8873451442432643 | ANKRD29; NOTCH1; ESPNL; |
| HSPV011b | | | | ANKRD33B; BCL3; PRICKLE2; |
| (ankyrin | | | | ANKEF1; PLA2G6; ANKRD60 |
| repeats) | | | | |
| Human | 2/20 | 0.05902612374508876 | 5.509142053445851 | CD274; NECTIN1 |
| adenovirus 61 | | | | |
| L5 Fiber | | | | |

### Conclusion

The majority of viruses enriched in interaction with the genes induced by ML228 are recognised as viral pathogens implicated in neurological infection and neuropathology, particularly in immunocompromised patients. It is evident from the results presented in table 2-4 that the ML228 treatment and thereby the activation of the HIF1-alpha-IRF1 antiviral axis induces the expression of many human genes which interact with several neurotropic viral proteins. Hence, a compound, such as ML228, can be used in the treatment or prophylaxis of a neurotropic viral infection caused by neurotropic viruses.

### REFERENCES

Jakub Treml et al., “Natural Products-Derived Chemicals: Breaking Barriers to Novel Anti-HSV Drug Development”, Viruses, 2020, 12, 154.

1. A method of treatment or prophylaxis of a neurotropic viral infection caused by a neurotropic virus, wherein said treatment comprises administering to a subject a compound which is an activator of hypoxia-inducible factor 1-alpha (HIF1-alpha) of formula (I): 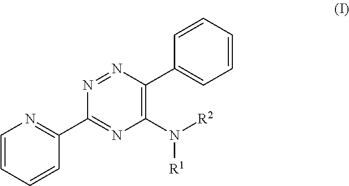
wherein
R¹ is represented by —(CH₂)ₙ—R³, or forms together with R² a five or
six-membered nitrogen containing heterocycle,
R² is selected from the group consisting of hydrogen and C₁-C₄ alkyl,
or forms together with R¹ a five- or six-membered nitrogen containing heterocycle,
n is an integer selected from 0, 1, 2 and 3, and
R³ is selected from the group consisting of optionally substituted
phenyl, allyl, methyl, cyclopropyl, cyclobutyl, cyclopentyl, cyclohexyl, tert-butyl, tetrahydrofuranyl, tetrahydropyranyl, and piperidinyl, and wherein the neurotropic virus is selected from the group consisting of human alphaherpesvirus 1 (HHV-1), human alphaherpesvirus 2 (HHV-2), human alphaherpesvirus 3 (HHV-3), human betaherpesvirus 5 (HHV-5), human betaherpesvirus 6A (HHV-6A), human betaherpesvirus 6B (HHV-6B), human betaherpesvirus 7 (HHV-7), human gammaherpesvirus 8 (HHV-8), human adenovirus (HAdV), Monkeypox virus Zaire-96-I-16, Human mastadenovirus, Vaccinia virus, horsepox virus HSPV050, cowpox virus, variola virus, human enterovirus, human rhinovirus, human papillomavirus, severe acute respiratory syndrome-related coronavirus 2 (SARS-CoV-2), encephalomyocarditis virus (EMCV), poliovirus, influenza A virus, influenza B virus, human immunodeficiency virus 1 (HIV-1), human immunodeficiency virus 2 (HIV-2), vesicular stomatitis Indiana virus (VSV or VSIV), and rabies lyssavirus.

2. The method according to claim 1, wherein the optionally substituted phenyl is substituted with one or more of the substituents selected from the list consisting of phenyl, C₁-C₄ alkyl, fluoro, chloro, and C₁-C₄ alkoxy.

3. The method according to claim 1, wherein the neurotropic virus is classified as a member of a family selected from the group consisting of herpesviridae, coronaviridae, picornaviridae, orthomyxoviridae, retroviridae, rhabdoviridae, flaviviridae, togaviridae, polyomaviridae, paramyxoviridae, peribunyaviridae, and matonaviridae.

4. The method according to claim 1, wherein the neurotropic virus is selected from the group consisting of herpes simplex virus 1 (HSV-1), herpes simplex virus 2 (HSV-2), encephalomyocarditis virus (EMCV), poliovirus, severe acute respiratory syndrome-related coronavirus 2 (SARS-CoV-2), influenza A virus, influenza B virus, human immunodeficiency virus 1 (HIV-1), human immunodeficiency virus 2 (HIV-2), vesicular stomatitis Indiana virus (VSV or VSIV), and rabies lyssavirus.

5. The method according to claim 1, wherein a disease caused by the viral infection is also treated or prevented.

6. The method according to claim 1, wherein the disease caused by the viral infection is selected from the group consisting of Alzheimer's disease, Alzheimer's disease related dementias (ADRD), Parkinson's disease, Guillain-Barre syndrome, multiple sclerosis, epilepsy, meningitis, aseptic meningitis, encephalitis, myelitis, acute disseminated encephalomyelitis, meningoencephalitis, herpes simplex encephalitis, recurrent genital herpes, varicella-zoster encephalitis, poliomyelitis, encephalomyocarditis, arthropod-borne encephalitis, subacute sclerosing panencephalitis (SSPE), progressive multifocal leukoencephalopathy (PML), flaccid paralysis, enteroviral disease, eastern equine encephalitis (EEE), western equine encephalitis, St. Louis encephalitis, rabies, La crosse encephalitis, progressive rubella panencephalitis (PRP), COVID-19, post-acute sequelae of COVID-19 (PASC), myalgic encephalomyelitis/chronic fatigue syndrome (ME/CFS), neuromyelitis optica spectrum disorder (NMOSD), dysautonomina, polyradiculitis, inflammatory neuropathies, and hypoxia.

7. The method according to claim 1, wherein the compound is administered during a latency period, or during an incubation period, or during a disease period, of the viral infection.

8. The method according to claim 1, wherein the compound is administered to a subject in need thereof.

9. The method according to claim 8, wherein the subject is a mammal.

10. The method according to claim 9, wherein the mammal is selected from the group consisting of human, pig, dog, horse, cattle, and cat; preferably a human.

11. The method according to claim 1, wherein the compound is administered to the subject by intravenous administration (IV), oral administration, intramuscular injection (IM), intrathecal administration, intraperitoneal injection (IP), and intraventricular administration.

12. The method according to claim 1, wherein the compound is administered in conjunction with at least one pharmaceutically acceptable excipient and/or pharmaceutically acceptable carrier.

13. The method according to claim 1, wherein the compound is administered in a lipid-based drug delivery systems (LBDDS).
